(19) United States
(12) Patent Application Publication
(10) Pub. No.: US 2005/0251061 A1
(43) Pub. Date: Nov. 10, 2005

(54) Method and system to record, store and transmit waveform signals to regulate body organ function

(76) Inventors: Eleanor L. Schuler, Rio Rancho, NM (US); Claude Lee, Reno, NV (US); Dennis P. Vik, Alburquerque, NM (US); Dennis E. Meyer, Alburquerque, NM (US)

Correspondence Address: Ralph C. Francis;Francis Law Group, 1942 Embarcadero, Oakland, CA, 94606, US

(21) Appl. No.: 11/125,480

(22) Filed: May 9, 2005

Related U.S. Application Data
(63) Continuation-in-part of application No. 10/000,005, filed on Nov. 20, 2001.
(60) Provisional application No. 60/249,882, filed on Nov. 20, 2000.

(51) Int. Cl. A61B 5/04; G11C 5/00; G10H 7/00

(52) U.S. Cl. 600/545

(57) ABSTRACT

A method to record, store and transmit waveform signals to regulate body organ function generally comprising capturing waveform signals that are generated in a subject's body and are operative in the regulation of body organ function and transmitting at least a first waveform signal to the body that is recognizable by at least one body organ as a modulation signal.

### CROSS-REFERENCE TO RELATED APPLICATIONS

This application is a continuation-in-part of U.S. application Ser. No. 10/000,005, filed Nov. 20, 2001.

### FIELD OF THE PRESENT INVENTION

The present invention relates generally to medical methods and systems for the treatment and/or management of body organs. More particularly, the invention relates to a method and system for recording, storing and transmitting waveform signals to regulate body organ function.

### BACKGROUND OF THE INVENTION

As is well known in the art, the brain modulates (or controls) body organ function via electrical signals (i.e., action potentials or waveform signals), which are transmitted through the nervous system. The nervous system includes two components: the central nervous system, which comprises the brain and the spinal cord, and the peripheral nervous system, which generally comprises groups of nerve cells (i.e., neurons) and peripheral nerves that lie outside the brain and spinal cord. The two systems are anatomically separate, but functionally interconnected.

Referring to FIG. 1, the nervous system comprises seven anatomical regions: (i) the spinal cord, (ii) the medulla, (iii) the pons, (iv) the cerebellum, (v) the midbrain, (vi) the diencephalon, and (vii) the cerebral hemisphere.

The spinal cord, which is subdivided into cervical, thoracic, lumbar, and sacral regions, is the most caudal part of the central nervous system. The spinal cord receives and processes sensory information from the skin, joints and muscles of the limbs and trunk. The spinal cord also controls movement of the limbs and trunk.

The spinal cord continues rostrally as the brain stem, which conveys information to and from the spinal cord and brain. The brain stem contains several distinct clusters of cell bodies, referred to as the cranial nerve nuclei. Some of the cranial nerve nuclei receive information from the skin and muscles of the head; others control motor output to muscles of the face, neck, and eyes. Still others are specialized for information from the special senses, e.g., hearing and taste. The brain stem also regulates levels of arousal and awareness through the diffusely organized reticular formation.

As illustrated in FIG. 1, the brain stem includes three regions: the medulla, pons and midbrain. The medulla oblongata, which lies directly above the spinal cord, includes several centers responsible for such vital automatic functions as digestion, breathing, and the control of heart rate. The pons, which lies above the medulla, conveys information about movement from the cerebral hemisphere to the cerebellum.

The cerebellum, which lies behind the pons, is connected to the brain stem by several major fiber tracts referred to as peduncles. The cerebellum modulates the force and range of movement.

The diencephalon, which lies rostral to the midbrain, contains two structures: the thalamus, which processes most of the information reaching the cerebral cortex from the rest of the central nervous system, and the hypothalamus, which regulates autonomic, endocrine and visceral function.

The cerebral hemisphere comprises the cerebral cortex and three deep-lying structures: the basal ganglia, the hippocampus and the amygdaloid nucleus. The basal ganglia are operative in regulating motor performance; the hippocampus is operative in various aspects of memory storage; and the amygdaloid nucleus coordinates autonomic and endocrine responses in conjunction with emotional states.

The peripheral nervous system includes somatic and autonomic divisions. The somatic division provides the central nervous system with sensory information relating to muscle and limb position and the external environment. The somatic division includes sensory neurons of the dorsal root and cranial ganglia that innervate the skin, muscles and joints.

Somatic motor neurons, which innervate skeletal muscle, have axons that project to the periphery. These axons are often considered part of the somatic division, even though the cell bodies are located in the central nervous system.

The autonomic division, which is often referred to as the autonomic motor system, is the motor system for the viscera, the smooth muscles of the body and the exocrine glands. The autonomic divisions comprise three spatially segregated subdivisions: the sympathetic, the parasympathetic and enteric nervous systems. The sympathetic system participates in the response of the body to stress, whereas the parasympathetic system acts to conserve the body's resources, e.g., restore the body to the resting state.

As indicated, the nervous system is constructed of nerve cells (or neurons) and glial cells (or glia), which support the neurons. Operative neuron units that carry signals from the brain are referred to as “efferent” nerves. “Afferent” nerves are those that carry sensor or status information to the brain.

Referring now to FIG. 2, there is shown an illustration of the links effected by the long nerves outside the central nervous system. As illustrated in FIG. 2, a typical neuron includes four morphologically defined regions: (i) cell body, (ii) dendrites, (iii) axon and (iv) presynaptic terminals. The cell body (soma) is the metabolic center of the cell. The cell body contains the nucleus, which stores the genes of the cell, and the rough and smooth endoplasmic reticulum, which synthesizes the proteins of the cell.

The cell body typically includes two types of outgrowths (or processes); the dendrites and the axon. Most neurons have several dendrites; these branch out in tree-like fashion and serve as the main apparatus for receiving signals from other nerve cells.

The axon is the main conducting unit of the neuron. The axon is capable of conveying electrical signals along distances that range from as short as 0.1 mm to as long as 2 m. Many axons split into several branches, thereby conveying information to different targets.

Near the end of the axon, the axon is divided into fine branches that make contact with other neurons. The point of contact is referred to as a synapse. The cell transmitting a signal is called the presynaptic cell, and the cell receiving the signal is referred to as the postsynaptic cell. Specialized swellings on the axon's branches (i.e., presynaptic terminals) serve as the transmitting site in the presynaptic cell.

Most axons terminate near a postsynaptic neuron's dendrites. However, communication can also occur at the cell body or, less often, at the initial segment or terminal portion of the axon of the postsynaptic cell.

The electrical signals transmitted along the axon, referred to as action potentials, are rapid and transient “all-or-none” nerve impulses. Action potentials typically have an amplitude of approximately 100 millivolts (mV) and a duration of approximately 1 msec. Action potentials are conducted along the axon, without failure or distortion, at rates in the range of approximately 1-100 meters/sec. The amplitude of the action potential remains constant throughout the axon, since the impulse is continually regenerated as it traverses the axon.

To ensure high-speed conduction of action potentials, large axons are surrounded by a fatty insulating sheath referred to as myelin. The myelin is interrupted at regular intervals by the nodes of Ranvier. It is at these nodes that the action potentials are regenerated.

A “neurosignal” is a composite signal that includes many action potentials. The neurosignal also includes an instruction set for proper organ function. By way of example, an instruction set for the diaphragm to perform an efficient ventilation will include information regarding frequency, initial muscle tension, degree (or depth) of muscle movement, etc.

Neurosignals are thus codes that contain complete sets of information for complete organ function. These codes must be “decoded” to be understood or executed by a target organ. The present technology, described in detail herein, establishes that the neurosignals contain more accurate and complete information than previously accepted.

Once these neurosignals, which are embodied in the “waveform signals” referred to herein, have been isolated, recorded, standardized and transmitted to a subject (or patient), the generated nerve-specific waveform instruction (i.e., waveform signal(s)) can be employed to, for example, restore breathing, restart hearts, eliminate pain, reduce blood pressure, restore sexual function, regulate bladder and bowel functions, reduce weight, move appendages, such as legs and arms, and wet dry eyes, via implants or transdermally, without harmful additional voltage or current.

In a recent study, phrenic neurosignals were collected from a rat and stored in a Neuriac® system. The neurosignals were subsequently transmitted to a dog (i.e., beagle) to control the diaphragm muscles, without added voltage, current, or modifying the signals.

The noted study thus establishes that neurocode similarity exists between various, and most likely all, common mammalian species. It is thus reasonable to conclude that neurosignals (and, hence, waveform signals embodying same) can be used to control the human respiratory system and, inferentially, other body functions.

Applicants have further found that existing models of nervous system communication are incomplete with regards to the description of functions which appear to be performed peripheral to the central nervous system. The operation of the long nerves has also been simplistically described as a physically mapped communication system. Further, the role served by ganglia, wherein nerve bodies are found in clumps along nerves, has not been clearly described.

It has been found that neural codes do, in fact, exist. The existence of neural codes thus requires the existence of decoders to ensure that peripheral function commands are interpreted and directed to the proper effectors. A model which explains this decoding function is shown in FIG. 24.

FIG. 24 shows a classical serial digital decoder formed by a delay line (a), an input “and” gate (b) and two inverters(c). As digital data, represented by “1” or “0”, is sent down the delay line, the conditions necessary for the “and” gate to have all input values “1” exist only when the sequence 11010 is sent into the delay line. Only this condition will result in a “1” being generated by the “and” gate; that is the gate has decoded the digital sequence required. An analog of each of these elements exists within a ganglion, wherein lie axons and terminal dendrites (delay line), excitatory and inhibitory terminal fibers (non-inverting and inverting inputs), and inter-neurons (and gates).

Accordingly, by simple mapping of inhibitory and excitatory synapses, the inter-neuron can be “programmed” to be either a serial or parallel decoder—sending a functional signal only when the digital pulses (axon potential pulses) arrive at the inter-neuron inputs simultaneously in the proper quantity and spacing.

Various apparatus, systems and methods have been developed, which include an apparatus for or step of recording action potentials or signals, to regulate body organ function. The signals are, however, typically subjected to extensive processing and are subsequently employed to regulate a “mechanical” device or system, such as a ventilator or prosthesis. Illustrative are the systems disclosed in U.S. Pat. Nos. 6,360,740 and 6,522,926.

In U.S. Pat. No. 6,360,740, a system and method for providing respiratory assistance is disclosed. The noted method includes the step of recording “breathing signals”, which are generated in the respiratory center of a patient. The “breathing signals” are processed and employed to control a muscle stimulation apparatus or ventilator.

In U.S. Pat. No. 6,522,926, a system and method for regulating cardiovascular function is disclosed. The noted system includes a sensor adapted to record a signal indicative of a cardiovascular function. The system then generates a control signal (as a function of the recorded signal), which activates, deactivates or otherwise modulates a baroreceptor activation device.

A major drawback associated with the systems and methods disclosed in the noted patents, as well as most known systems, is that the control signals that are generated and transmitted are “user determined” and “device determinative”. The noted “control signals” are thus not related to or representative of the signals that are generated in the body and, hence, would not be operative in the control or modulation of a body organ function if transmitted directly thereto.

It would thus be desirable to provide a method and system for regulating body organ function that includes means for recording waveform signals that are generated in the body, means for storing the collected waveform signals, and means for providing and transmitting waveform signals directly to the body that substantially correspond to the recorded waveform signals and are operative in the control of body organ function.

It is therefore an object of the present invention to provide a method and system for regulating body organ function that overcomes the drawbacks associated with prior art methods and systems for regulating body organ function.

It is another object of the invention to provide a method and system for regulating body organ function that includes means for recording waveform signals that are generated in the body.

It is another object of the invention to provide a method and system for regulating body organ function that includes means for generating signals that substantially correspond to waveform signals that are generated in the body and are operative in the control of body organ function.

It is another object of the invention to provide a method and system for regulating body organ function that includes processing means adapted to generate a base-line signal that is representative of at least one waveform signal generated in the body from recorded waveform signals.

It is another object of the invention to provide a method and system for regulating body organ function that includes processing means adapted to compare recorded waveform signals to baseline signals and generate a modified base-line signal as a function of the recorded waveform signal.

It is another object of the invention to provide a method and system for regulating body organ function that can be readily employed in the assessment and/or treatment of multiple disorders, including, but not limited to, sleep apnea, respiratory distress, asthma, acute low blood pressure, abnormal heart beat, paralysis, spinal chord injuries, acid reflux, obesity, erectile dysfunction, a stroke, tension headaches, a weakened immune system, irritable bowl syndrome, low sperm count, sexual unresponsiveness, muscle cramps, insomnia, incontinence, constipation, nausea, spasticity, dry eyes syndrome, dry mouth syndrome, depression, epilepsy, low levels of growth hormone and insulin, abnormal levels of thyroid hormone, melatonin, adrenocorticotropic hormone, ADH, parathyroid hormone, epinephrine, glucagon and sex hormones, pain block and/or abatement, physical therapy and deep tissue injury.

It is another object of the invention to provide a method and system for regulating body organ function that includes means for transmitting signals directly to the body that substantially correspond to waveform signals that are generated in the body and are operative in the control of body organ function.

It is another object of the present invention to provide a method and system for regulating body organ function that includes means for transmitting signals directly to the nervous system in the body that substantially correspond to waveform signals that are generated in the body and are operative in the control of body organ function.

### SUMMARY OF THE INVENTION

In accordance with the above objects and those that will be mentioned and will become apparent below, the method to record, store and transmit waveform signals to regulate body organ function generally comprises (i) capturing waveform signals that are generated in a subject's body and are operative in the regulation of body organ function and (ii) transmitting at least a first waveform signal to the body that is recognizable by at least one body organ as a modulation signal.

In one embodiment of the invention, the first waveform signal includes at least a second waveform signal that substantially corresponds to at least one of the captured waveform signals and is operative in the regulation of the body organ.

In one embodiment of the invention, the first waveform signal is transmitted to the subject's nervous system.

In another embodiment, the first waveform signal is transmitted proximate to the body organ.

In another embodiment of the invention, the method to record, store and transmit waveform signals to regulate body organ function generally comprises (i) capturing waveform signals that are generated in the body and are operative in the regulation of body organ function and (ii) storing the captured waveform signals in a storage medium, the storage medium being adapted to store the captured waveform signals according to the organ regulated by the captured waveform signals, and (iii) transmitting at least a first waveform signal to the body that substantially corresponds to at least one of the captured waveform signals and is operative in the regulation of at least one body organ.

In one embodiment of the invention, the storage medium is further adapted to store the captured waveform signals according to the function performed by the captured waveform signals.

In another embodiment of the invention, the method to record, store and transmit waveform signals to regulate body organ function generally comprises (i) capturing a first plurality of waveform signals generated in a first subject's body, the first plurality of waveform signals including first waveform signals that are operative in the control of a first body organ, (ii) generating a base-line waveform signal from the first waveform signals, (iii) capturing a second plurality of waveforms signals generated in the first subject's body, the second plurality of waveform signals including at least a second waveform signal that is operative in the control of the first body organ, (iv) comparing the base-line waveform signal to the second waveform signal, (v) generating a third waveform signal based on the comparison of the base-line and second waveform signals, and (vi) transmitting the third waveform signal proximate to the first body organ, the third waveform signal being operative in the regulation of the first body organ function.

In one embodiment of the invention, the first plurality of waveform signals is captured from a plurality of subjects.

Preferably, the third waveform signal is transmitted to said subject's nervous system.

In an alternative embodiment, the third waveform signal is transmitted proximate to the first body organ.

The system to record, store and transmit waveform signals to regulate body organ function in accordance with one embodiment of the invention generally comprises (i) at least a first signal probe adapted to capture waveform signals from a subject's body, the waveform signals being representative of waveform signals naturally generated in the body and indicative of body organ function, (ii) a processor in communication with the signal probe and adapted to receive the waveform signals, the processor being further adapted to generate at least a first waveform signal based on the captured waveform signals, the first waveform signal being recognizable by at least one body organ as a modulation signal and (iii) at least a second signal probe adapted to be in communication with the subject's body for transmitting the first waveform signal proximate to the body organ to regulate organ function.

In an alternative embodiment, the signal probe is positioned and adapted to transmit the first waveform signal to the subject's nervous system.

In one embodiment, the processor includes a pulse rate detector for sampling the captured waveform signals and a pulse rate generator for generating the first waveform signal.

Preferably, the processor includes a storage medium adapted to store the captured waveform signals.

Preferably, the storage medium is adapted to store the captured waveform signals according to the organ regulated by the captured waveform signals.

In one embodiment of the invention, the storage medium is further adapted to store the captured waveform signals according to the function performed by the captured waveform signals.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is an illustration of the central nervous system;

FIG. 2 is an illustration of the links effected by the long nerves outside the central nervous system;

FIGS. 3A and 3B are illustrations of waveform signals captured from the body that are operative in the control of the respiratory system;

FIGS. 4A through 4D are illustrations of waveform signals captured from the body that are operative in the control of the skeletal muscles of the arm, forearm, hands and fingers;

FIG. 5 is a perspective view of one embodiment of a signal probe, according to the invention;

FIG. 6A is a side elevation view of another embodiment of a signal probe, according to the invention;

FIG. 6B is a perspective view of the signal probe shown in FIG. 6A;

FIG. 7A is an illustration showing one embodiment of the engagement of the signal probes of the invention to a target nerve;

FIG. 7B is an illustration showing an alternative embodiment of the engagement of the a single signal probe of the invention to a target nerve;

FIG. 8 is a further illustration of the chest and diaphragm regions of a subject showing the engagement of the signal probes of the invention to the phrenic nerves;

FIG. 9 is a schematic illustration of one embodiment of the body organ regulation system, according to the invention;

FIGS. 10A-10B and 11A-11B are illustrations of waveform signals captured from the body that are operative in the control of the cardiovascular system;

FIGS. 12A and 12B are illustrations of waveform signals captured from the diaphragm muscle that are operative in the control of the respiratory system;

FIGS. 13A-13B and 14A-14B are illustrations of waveform signals captured from the phrenic nerve that are operative in the control of the respiratory system;

FIGS. 15A-15B and 16A-16B are illustrations of waveform signals captured from the body that are operative in the control of the shoulder muscle;

FIGS. 17A-17B and 18A-18B are illustrations of waveform signals captured from the radial nerve that are operative in the control of the muscles of the arm, wrist and fingers;

FIGS. 19A-19B and 20A-20B are illustrations of waveform signals captured from the sciatic nerve that are operative in the control of muscles in the leg, ankle and toes;

FIGS. 21A and 21B are illustrations of waveform signals captured from the ulnar nerve that are operative in the control of muscles in the arm, wrist and fingers;

FIG. 22 is a schematic illustration of the storage means of the invention;

FIGS. 23A and 23B are illustrations of waveform signals that have been generated by the process means of the invention; and

FIG. 24 is a schematic illustration of a prior art serial digital decoder.

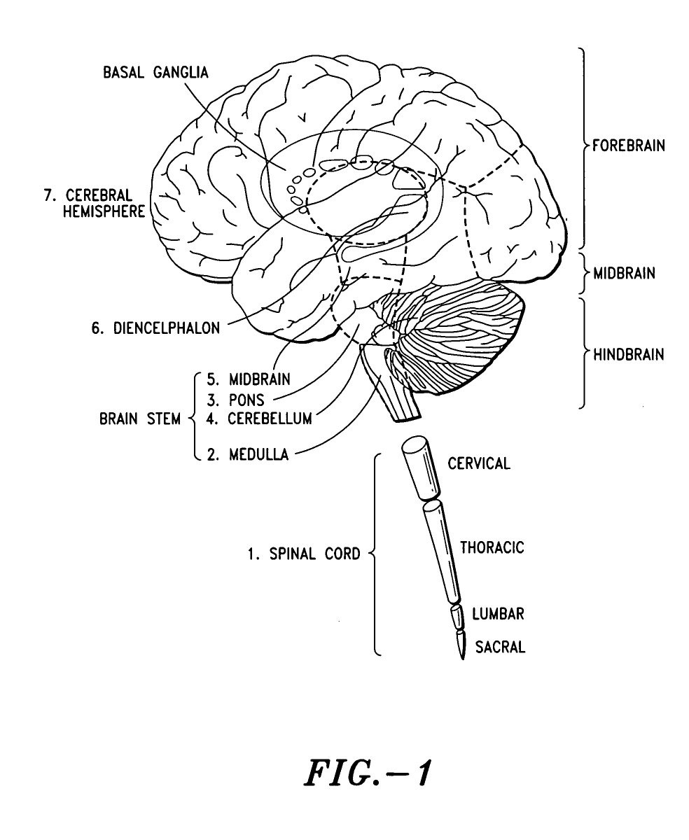

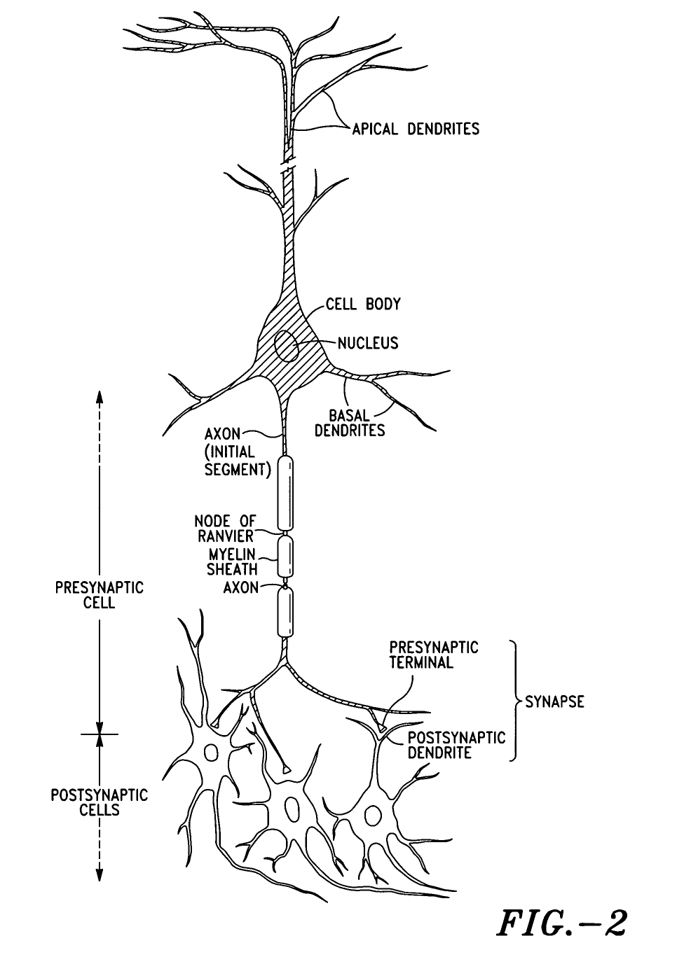

### DETAILED DESCRIPTION OF THE INVENTION

Before describing the present invention in detail, it is to be understood that this invention is not limited to particularly exemplified apparatus, systems, structures or methods as such may, of course, vary. Thus, although a number of apparatus, systems and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “a waveform signal” includes two or more such signals; reference to “a neuron” includes two or more such neurons and the like.

### Definitions

The term “nervous system”, as used herein, means and includes the central nervous system, including the spinal cord, medulla, pons, cerebellum, midbrain, diencephalon and cerebral hemisphere, and the peripheral nervous system, including the neurons and glia.

The terms “waveform” and “waveform signal”, as used herein, mean and include a composite electrical signal that is generated in the body and carried by neurons in the body, including neurocodes and components and segments thereof.

The term “body organ”, as used herein, means and includes, without limitation, skin, bones, cartilage, tendons, ligaments, skeletal muscles, smooth muscles, heart, blood vessels, brain, spinal cord, peripheral nerves, nose, eyes, ears, mouth, tongue, pharynx, larynx, trachea, bronchus, lungs, esophagus, stomach, liver, pancreas, gall bladder, small intestines, large intestines, rectum, anus, kidneys, ureter, bladder, urethra, hypothalamus, pituitary, thyroid, adrenal glands, parathyroid, pineal gland, ovaries, oviducts, uterus, vagina, mammary glands, testes, seminal vesicles, prostate, penis, lymph nodes, spleen, thymus and bone marrow.

The terms “patient” and “subject”, as used herein, mean and includes humans and animals.

The term “plexus”, as used herein, means and includes a branching or tangle of nerve fibers outside the central nervous system.

The term “ganglion”, as used herein, means and includes a group or groups of nerve cell bodies located outside the central nervous system.

The present invention substantially reduces or eliminates the disadvantages and drawbacks associated with prior art methods and systems for regulating body organ function. In one embodiment of the invention, the method and system for regulating body organ function generally comprises means for recording (or capturing) waveform signals that are generated in the body, means for storing the recorded waveform signals, means for generating at least one signal that substantially corresponds to at least one recorded waveform signal and is operative in the regulation of at least one body organ, and means for transmitting the signal to the body organ. Each of the noted components (or modules) is described in detail below.

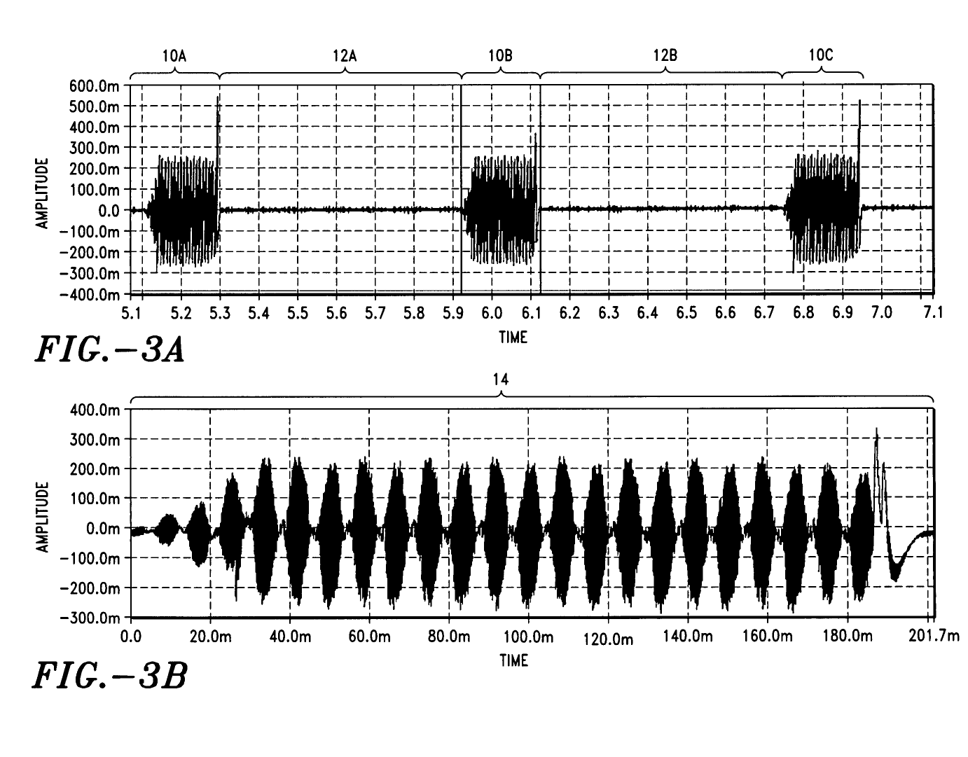

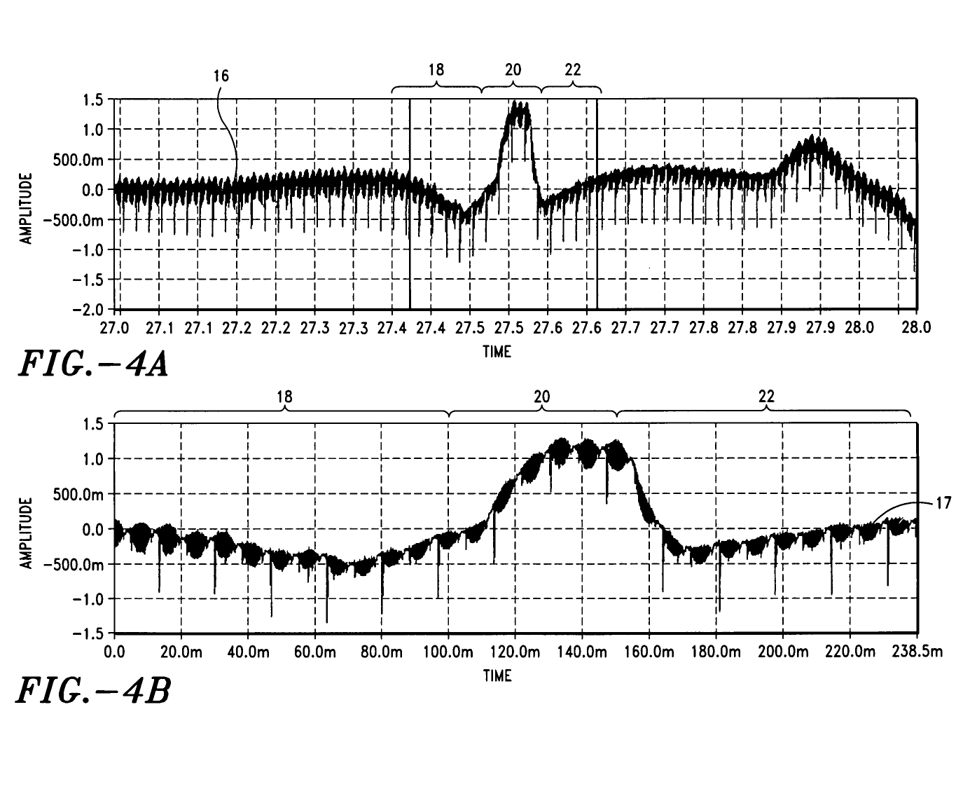

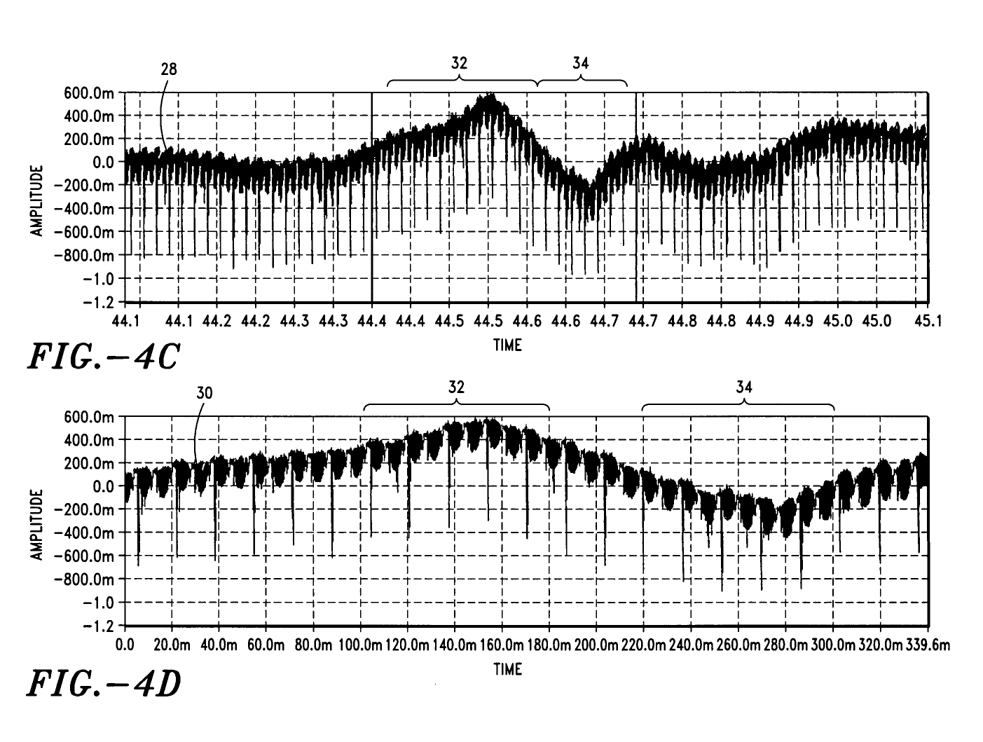

Referring first to FIGS. 3A-3B and 4A-4D, there are shown exemplar waveform signals operative in the regulation of the respiratory system and skeletal muscles, respectively.

FIGS. 3A and 3B represent actual waveform signals that are operative in the efferent operation of the human (and animal) diaphragm; FIG. 3A showing three (3) signals 10A, 10B, 10C, having rest periods 12A, 12B therebetween, and FIG. 3B showing an expanded view of signal 10B. The noted signals traverse the phrenic nerve, which runs between the cervical spine and the diaphragm.

As will be appreciated by one having skill in the art, signals 10A, 10B, 10C will vary as a function of various factors, such as physical exertion, reaction to changes in the environment, etc. As will also be appreciated by one having skill in the art, the presence, shape and number of pulses of signal segment 14 can similarly vary from muscle (or muscle group) signal-to-signal.

FIGS. 4A and 4B represent waveform signals that are operative in the control of the skeletal muscles of the arm, forearm, hands and fingers. The signals 16, 17 shown in FIGS. 4A and 4B bring the arm upward and pull the hand back with the fingers spread. The signals 28, 30 shown in FIGS. 4C and 4D provide the same movement as the signals shown in FIGS. 4A and 4B with less intensity (i.e., moderate movement).

As discussed in detail herein, each signal 16, 28 includes a negative segment 18, which is believed to reflect the muscle and/or nerve setting up for movement. Following the negative segment 18 is a large positive segment 20, 32, which produces the desired muscle movement, and a negative segment 22, 34 thereafter, reflecting the rest and evaluation segment of the signal.

Signal Acquistion

Various apparatus and methods have been described in the art and employed to capture waveform signals from the body. The conventional apparatus and methods typically communicate with the nerves via direct attachment of the apparatus (e.g., probe) to a target nerve. Illustrative are the probes manufactured by World Precision Instruments, and Harvard Apparatus, sold under the trade names Metal Electrodes Tungsten Profile B and Reusable Probe Point 28 gauge 9.5 mm length, respectively.

Conventional probes are, however, too large for certain mammalian applications, particularly, the nerves of a rat. As is known in the art, a rat phrenic nerve has a diameter of approximately 0.254 mm.

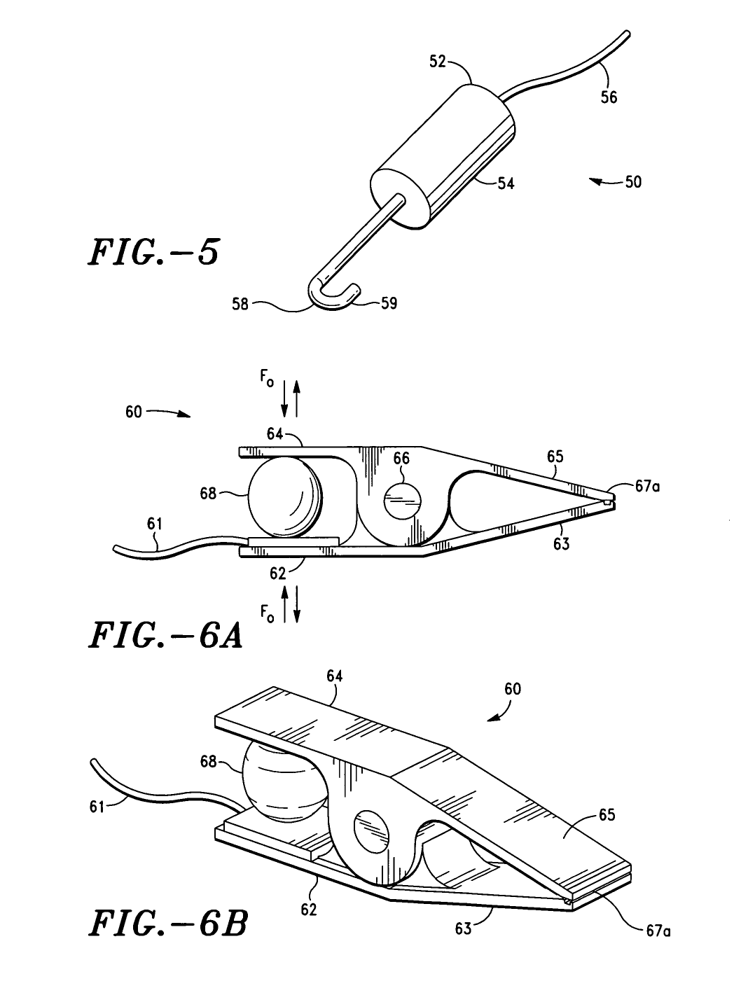

Novel nerve probes were thus developed and employed (in one embodiment of the invention) to capture signals directly from small diameter nerves. The noted probes are shown in FIGS. 5, 6A and 6B.

Referring first to FIG. 5, there is shown a “needle” probe 50, which is adapted to cradle a small target nerve. As illustrated in FIG. 5, the probe 50 includes an electrode 52, which is preferably encased in an insulated head 54, an electrical lead 56 and a hooked connecting member 58, which extends from the electrode 52.

In a preferred embodiment of the invention, the connecting member 58 comprises a fine wire having a diameter in the range of 0.02-0.4, more preferably, in the range of 0.03-0.26 mm. Preferably, the wire comprises silver, platinum or gold, or like material.

According to the invention, the connecting member 58 can be coated with various materials, such as non-conductive plastic, rubber or silicon rubber, to insulate the probe from the surrounding tissue. In a preferred embodiment, the connecting member 58 is coated with a non-conductive polymeric material.

Preferably, the connecting member 58 has a length in the range of 6.0-26 mm, more preferably, in the range of 7.5-15.25 mm. The hooked region 59 of the connecting member 58 preferably has a radius in the range of approximately 0.5-1.25 mm, more preferably, in the range of approximately 0.51-0.77 mm.

Referring now to FIGS. 6A and 6B, there is shown a further probe, designated generally 60, adapted to acquire signals from small, target nerves. As illustrated in FIGS. 6A and 6B, the probe 60 includes an electrical lead 61, a planar bottom section 62 and a planar top section 64, which is hingedly connected to the bottom section 62 via pin 66.

The top and bottom sections 62, 64 include nose regions 63, 65, respectively, which are designed and adapted to be proximate each other when the top and bottom sections 62, 64 are in a closed position. Disposed proximate the edge region of nose region 63 is a nerve channel 67a adapted to receive the target nerve.

The probe 60 further includes a force member 68 adapted to provide a closing force to return the top and bottom sections 62, 64 to the closed position. In a preferred embodiment, the force member 68 comprises a silicon rubber drop.

In operation, a force (designated Fₒ) is applied to the top and bottom sections 62, 64 proximate the end opposite the nose regions 63, 65 to open the probe 60. The target nerve is then placed in nerve channel 67a and the force (Fₒ) is released, whereby a closing force (F_{c}) is provided by the silicon rubber drop 68 and the nerve channel 67a seats the target nerve. Preferably, the closing force (F_{c}) is less than 0.5 kg, more preferably, approximately 0 kg, when the probe 60 is in a closed position.

As is well known in the art, direct attachment to a nerve typically requires preparation of the nerve to facilitate communication by and between the nerve and the probe. For example, in some techniques, all or a portion of the myelin is removed to expose the axon and, hence, provide an engagement region for attachment of the probe.

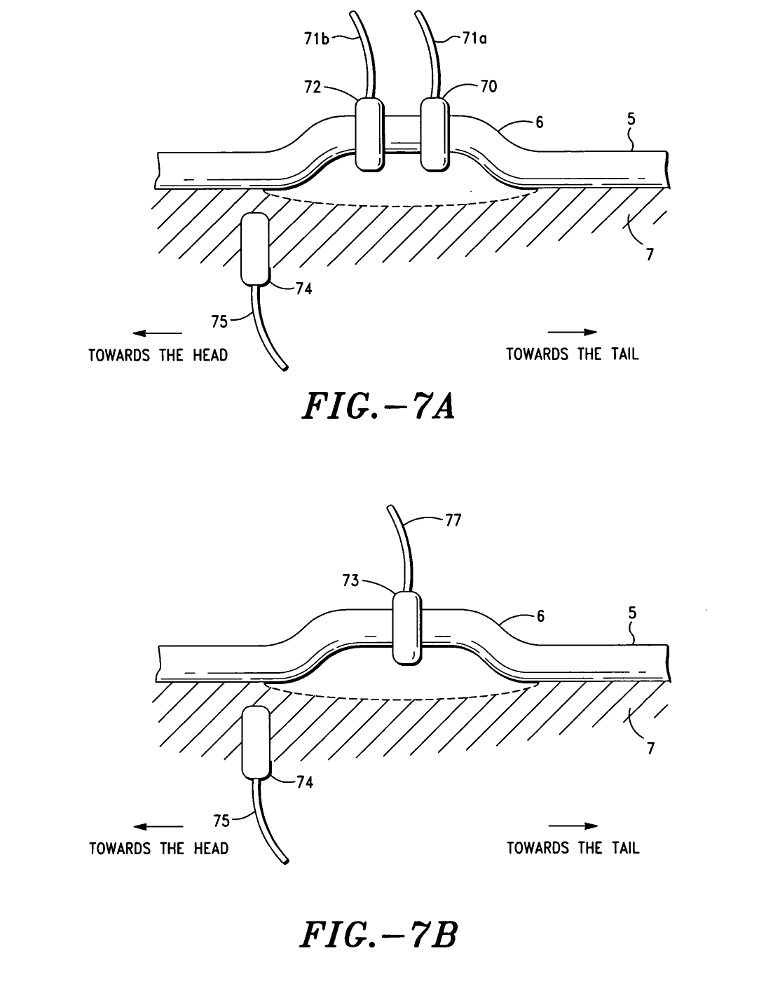

Applicants have, however, developed a technique to capture signals directly from a nerve that does not require damaging or altering the tissues of the nerve. As illustrated in FIGS. 7A and 7B, in a preferred embodiment of the invention, the target nerve (5) is merely separated from the surrounding tissue 7 (e.g., muscle, veins, connective tissue) and elevated slightly.

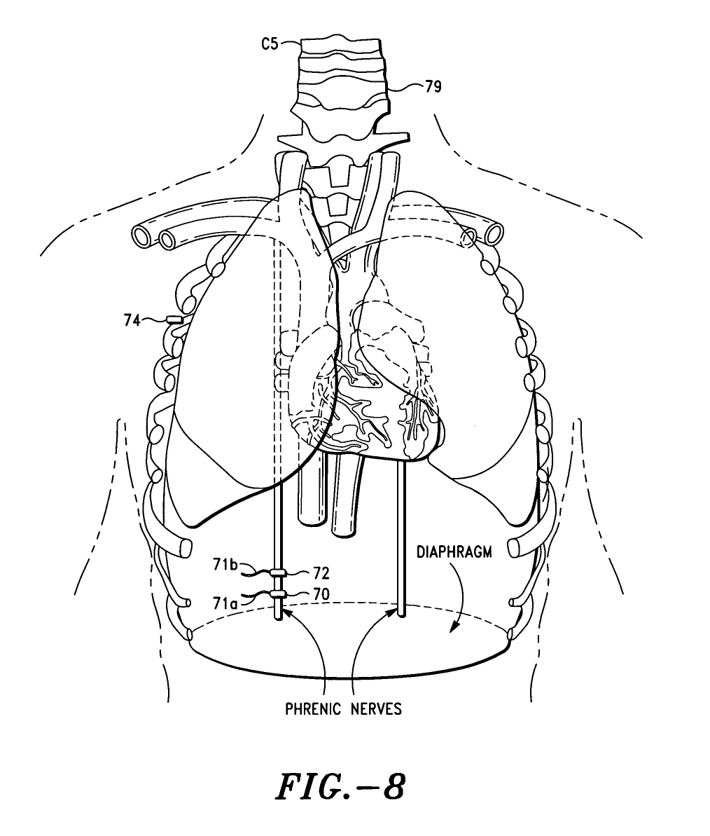

In one embodiment of the invention, a dual signal probe system, such as shown in FIGS. 7A and 8 is employed. In an alternative embodiment, shown in FIG. 7B, a single probe system is employed.

Referring now to FIGS. 7A and 8, in a preferred embodiment of the invention, a positive signal probe 70 and a negative signal probe 72 are secured to the target nerve 5 proximate the raised nerve area 6. Preferably, the positive probe 70 and negative probe 72 have a space therebetween in the range of 0.5-25 mm, more preferably, in the range of approximately 0.75-20 mm. It is also preferred that the probes 70, 72 are not in contact with any surrounding tissue.

As illustrated in FIG. 7A, a ground probe 74 is attached to an interior muscle. This creates an electrostatic shield using the subject's interior muscles.

Referring now to FIG. 7B, in an alternative embodiment, a single probe 73 is connected to the target nerve 5. The ground probe 74 is similarly attached to an interior muscle.

In an alternative embodiment of the invention, the target nerve is dissected to expose the afferent and efferent nerve bundles prior the placement of the probe (e.g., probe 60) or probes (e.g., probes 70, 72, 73) thereon. While this technique can, and in many instances will, damage the nerve, it would provide more definite afferent and efferent signals.

In further envisioned embodiments of the invention, the nerve is stimulated either directly or indirectly by electromagnetic, laser or sound waves, wherein the signal is captured by a receiving antenna that is in communication with a target nerve.

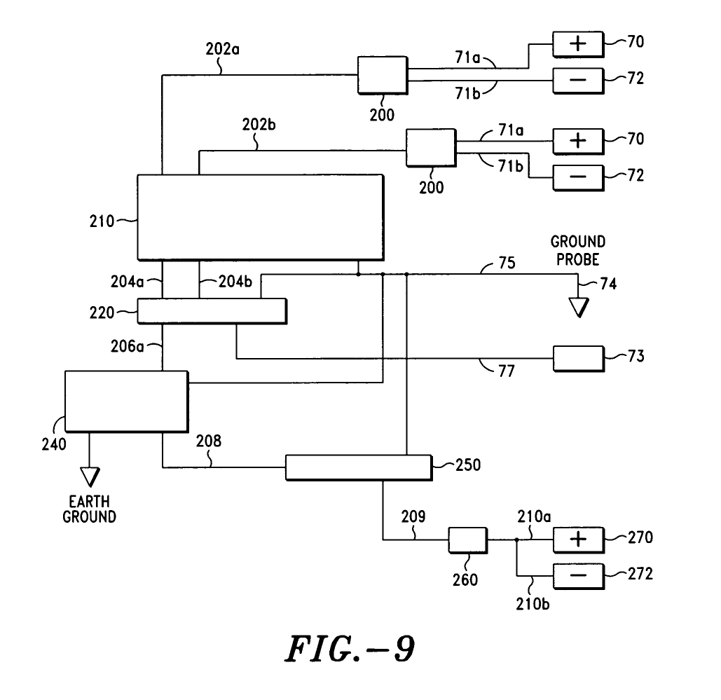

Referring now to FIG. 9, there is shown one embodiment of a system (or processor) for regulating body organ function. As illustrated in FIG. 9, the electrical leads 71a, 71b of the positive and negative “high speed” signal probes 70, 72, respectively, are preferably connected to a high impedance head-stage preamplifier 200. As will be appreciated as one having ordinary skill in the art, various pre-amps can be employed within the scope of the invention. In a preferred embodiment of the invention, the pre-amp 200 comprises a Super-Z high-impedance preamplifier manufactured by CWE, Inc.

As is known in the art, the noted preamplifier has a very high impedance, low drift, differential input amplifier and a built in DC off-set adjustment. The unit is preferably set to the AC (alternating current) mode, which eliminates any DC (direct current) off-sets. The amplifications of the unit are also preferably set to 0.

As illustrated in FIG. 9, the signal is routed from the high impedance head-stage pre-amplifier 200 to the bioamplifier 210 via leads 202a, 202b. The ground probe 74 is also in communication with the bioamplifier 210 via lead 75. In one embodiment, the bioamplifier 210 is preferably set to magnify the waveform signal X 50 to produce a desirable signal.

As will be appreciated by one skilled in the art, the captured signal(s) will include the waveform signal representative of the signal produced in the body as well as background noise and extraneous material. The captured signal is thus filtered to substantially reduce, more preferably, eliminate, the background noise and extraneous material.

According to the invention, various conventional apparatus and techniques can be employed to filter the captured signals. In a preferred embodiment, the signals are filtered by a 4 pole Butterworth filter with resultant attenuation of −12 dB/octave for frequencies outside of the selected cutoff frequencies.

Preferably, the high pass filter cutoff frequency is set to 1 Hz and the low pass filter cutoff frequency is preferably set to 10,000 Hz.

In one embodiment of the invention, the magnified signal is then transmitted (or routed) from the bioamplifier 210 to the analog to digital conversion unit 220, which is adapted to convert the signal from an analog format to a digital format. This conversion makes the waveform signal easy for the computer to display, read, and store by changing the wave of information into a stream of data points.

According to the invention, various analog to digital converters can be employed to provide the noted conversion. In a preferred embodiment of the invention, the conversion apparatus comprises a National Instruments Corporation unit (Part number DAQ Pad 6070E).

Referring back to FIG. 9, in an alternative embodiment of the invention, wherein a low speed input probe 73 is employed, the signal captured by the low speed probe 73 is routed directly to the analog digital converter 220 via lead 77. The ground probe 74 is similarly routed to the analog digital converter 220 via lead 75.

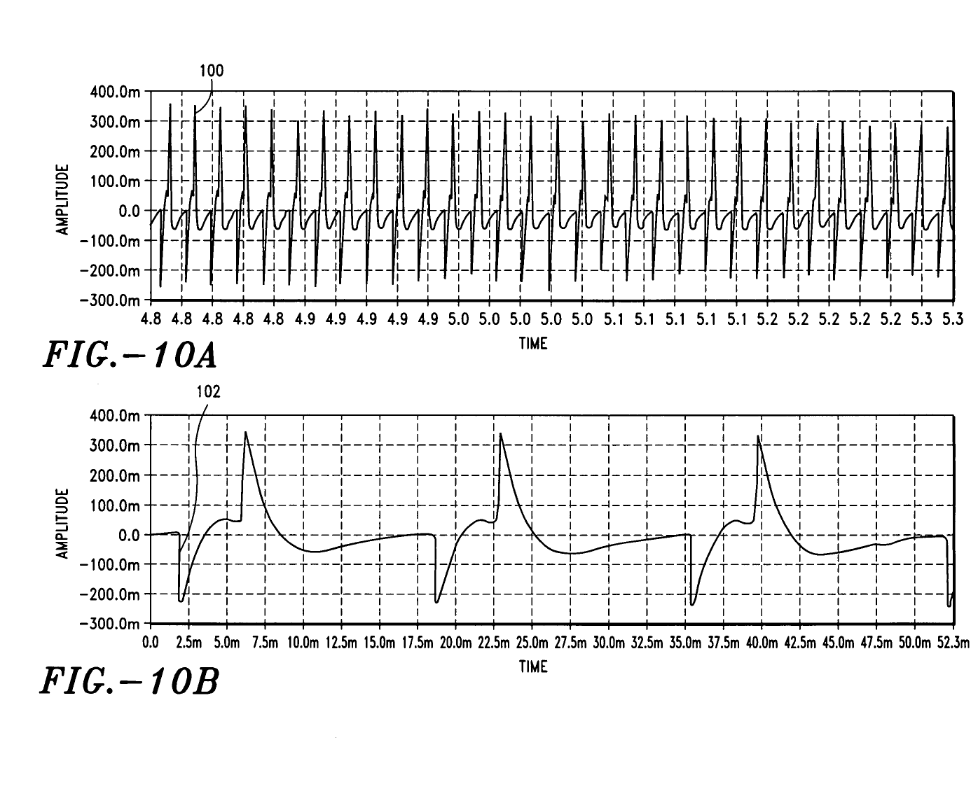

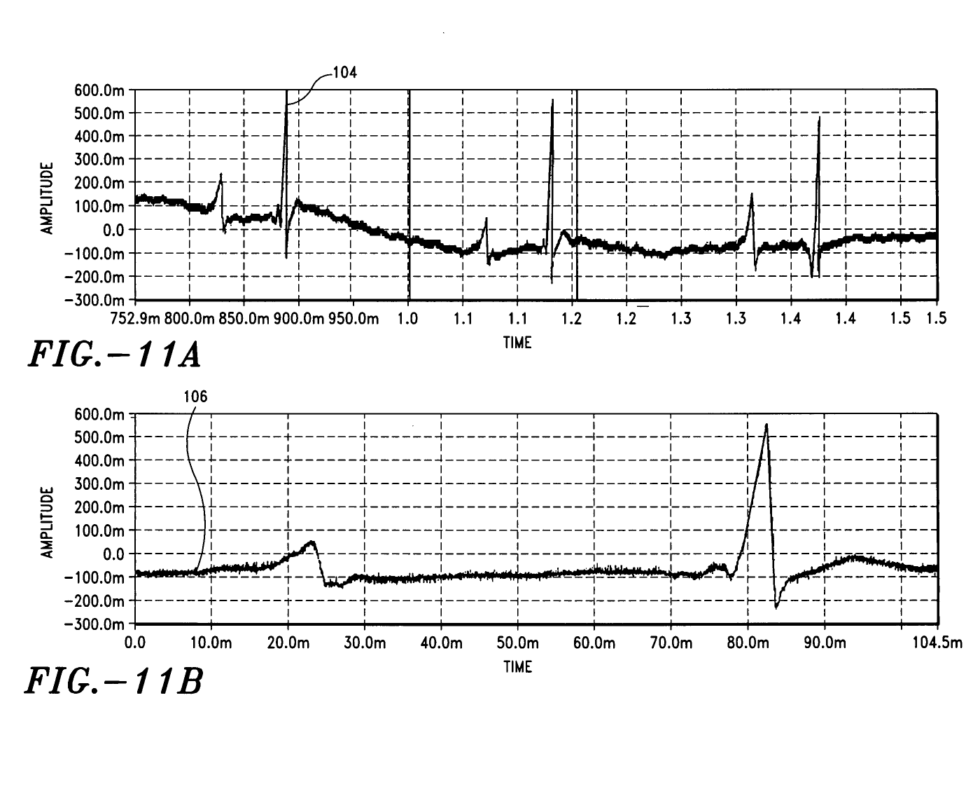

Referring now to FIGS. 10A-10B through FIGS. 21A-21B, there are shown various waveform signals that were captured (or recorded) from a subject (i.e., rat) using the apparatus and methods of the invention. Referring first to FIGS. 10A-10B and 11A-11B, there are shown signals 100, 102, 104, 106 acquired from the phrenic nerves that are operative in the control of the cardiovascular system (i.e., heart).

Signals 100, 102 reflect the normal heart rate of a rat. Signals 104, 106 reflect the heart rate of the rat under stress. The sample rate for the signals 100, 102, 104, 106 shown in FIGS. 10A-10B and 11A-11B were 10,000 point/sec. and 250,000 point/sec, respectively.

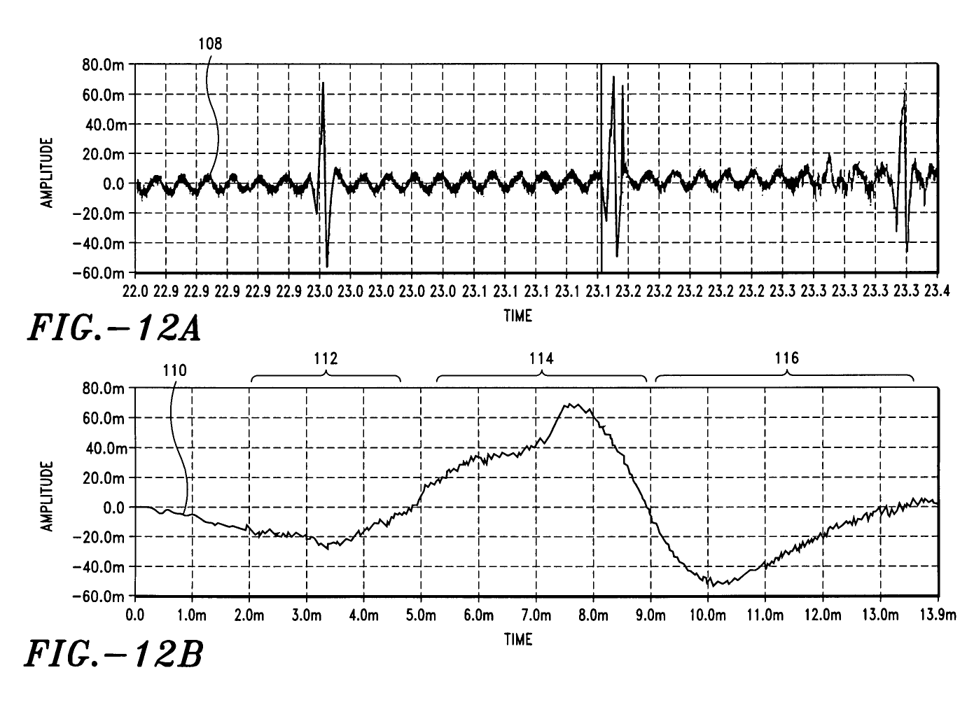

Referring now to FIGS. 12A-12B, there are shown signals 108 and 110 that were acquired directly from the diaphragm muscle that are operative in the control of the respiratory system. Referring to FIG. 12B, which is an expanded segment of signal 108, it can be seen that the signal 110 reflects a common muscle signal pattern. It can also be seen that the signal 110 has an initial negative region or segment (designated generally 112) followed by a sharp positive spike (designated generally 114), and a longer segment (designated generally 116) thereafter.

It is believed that the first negative segment 112 reflects the nerve and/or muscle setting up for the contraction. The large positive spike 114 is the signal segment that causes the muscle to contract. Changes in the amplitude of the positive segment 114 determine how much the muscle contracts. The longer negative segment 116 is believed to be the rest and evaluation portion of the signal.

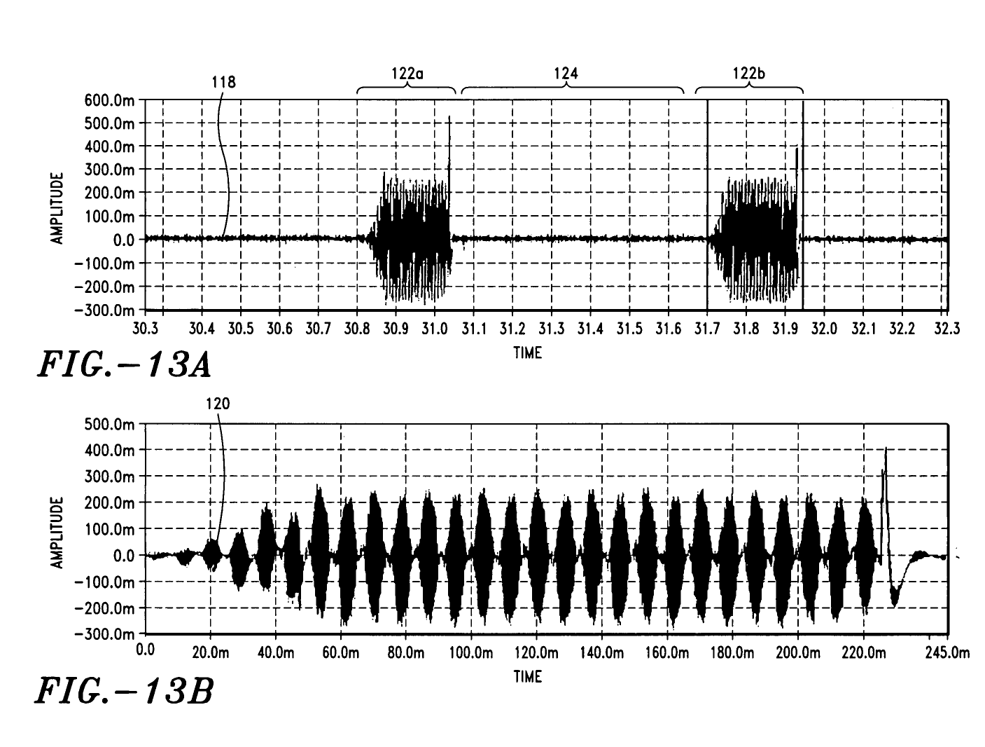

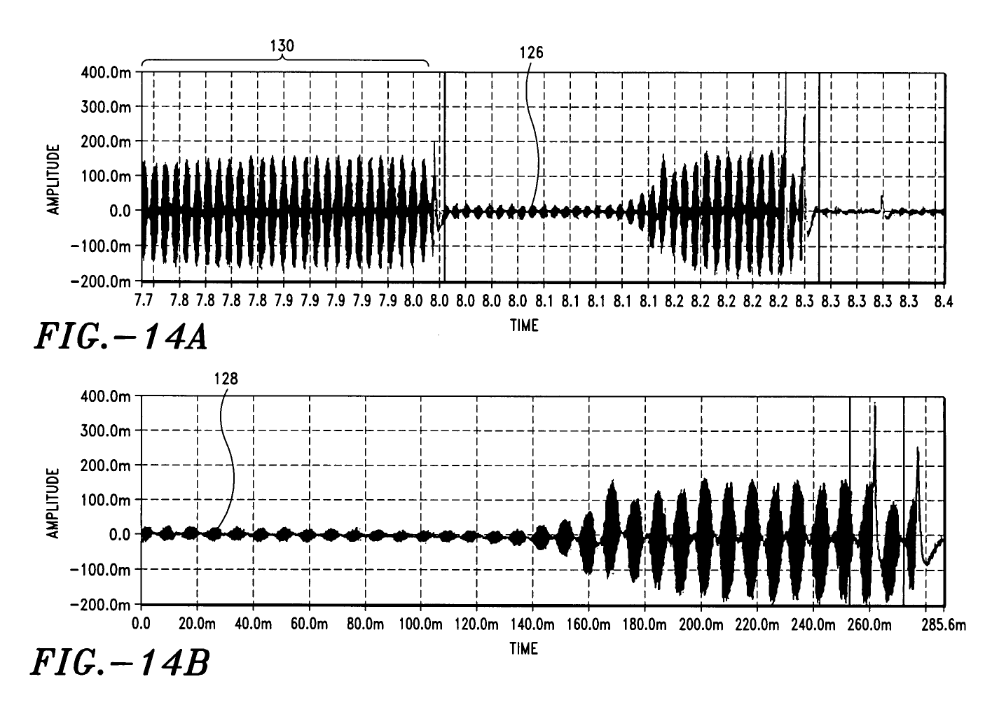

Referring now to FIGS. 13A-13B, and 14A-14B, there are shown traces 118, 120 having waveform signals 122A, 122B acquired from the phrenic nerve that are operative in the control of the respiratory system. FIG. 13A shows the two signals 122A, 122B having a rest period 124 therebetween. FIG. 13B shows an expanded view of signal 122B.

Referring now to FIGS. 14A-14B, there are shown signals 126, 128, which reflect a rat in distress (i.e., going into shock). Referring to FIG. 13A, it can be seen that the pattern of the signal 126 has changed greatly as the rat tries to breathe rapidly. In segment 130 of signal 126 it can be seen that the initial segment is longer and the number of pulses is greater.

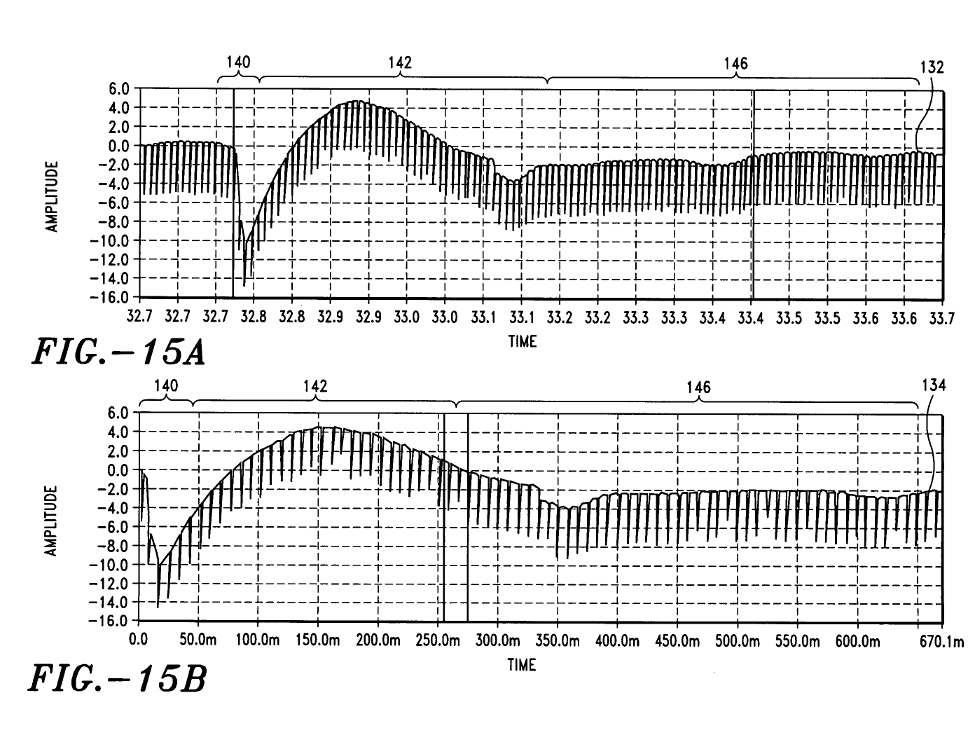

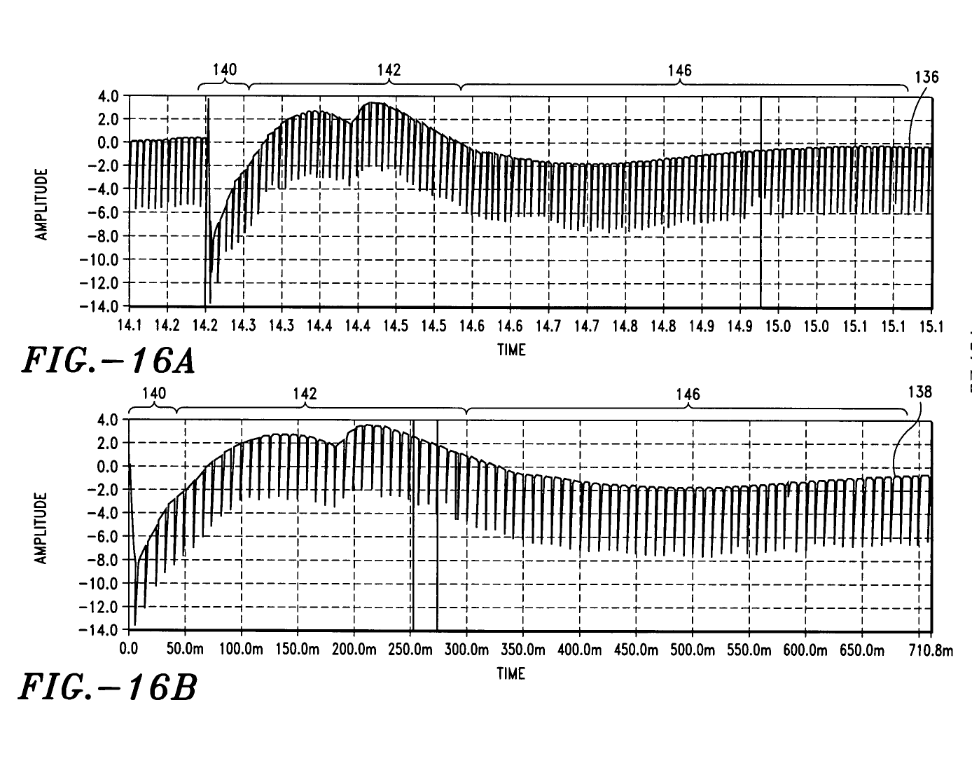

Referring now to FIGS. 15A-15B and 16A-16B, there are shown signals 132, 134, 136 and 138 acquired from the suprascapular nerve that are operative in the control of a shoulder muscle. The noted signals 132, 134, 136, 138 similarly reflect the common signal pattern for muscle movement.

Each signal 132, 134, 136, 138 includes a sharp negative segment 140, which is believed to reflect the muscle and/or nerve setting up for movement. Following the negative segment 140, there is a large positive segment 142. The shape of this segment 142 will change based upon how fast or smoothly the muscle is to move. The last segment 146 is believed to be the rest and evaluation portion of the signal.

As with most signals, the longer the positive segment 142, the longer and more pronounced the muscle movement. The shorter the segment 142, the quicker and shorter the muscle movement. The strength of the muscle movement is also dependent on the amplitude of the signal, i.e., higher voltages cause stronger movement.

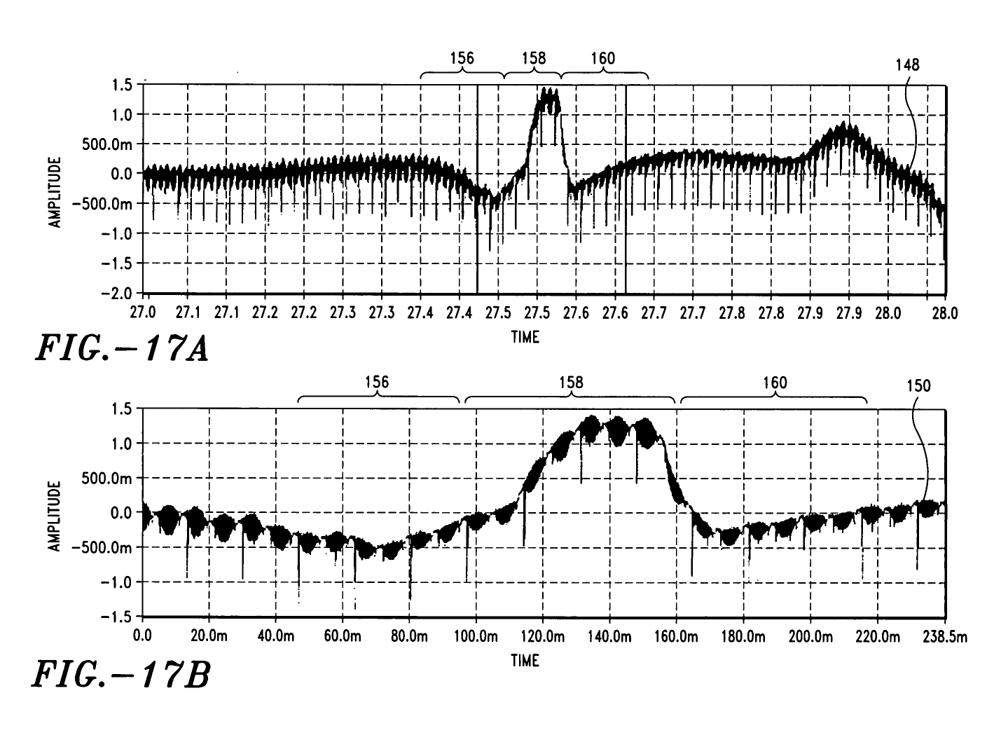

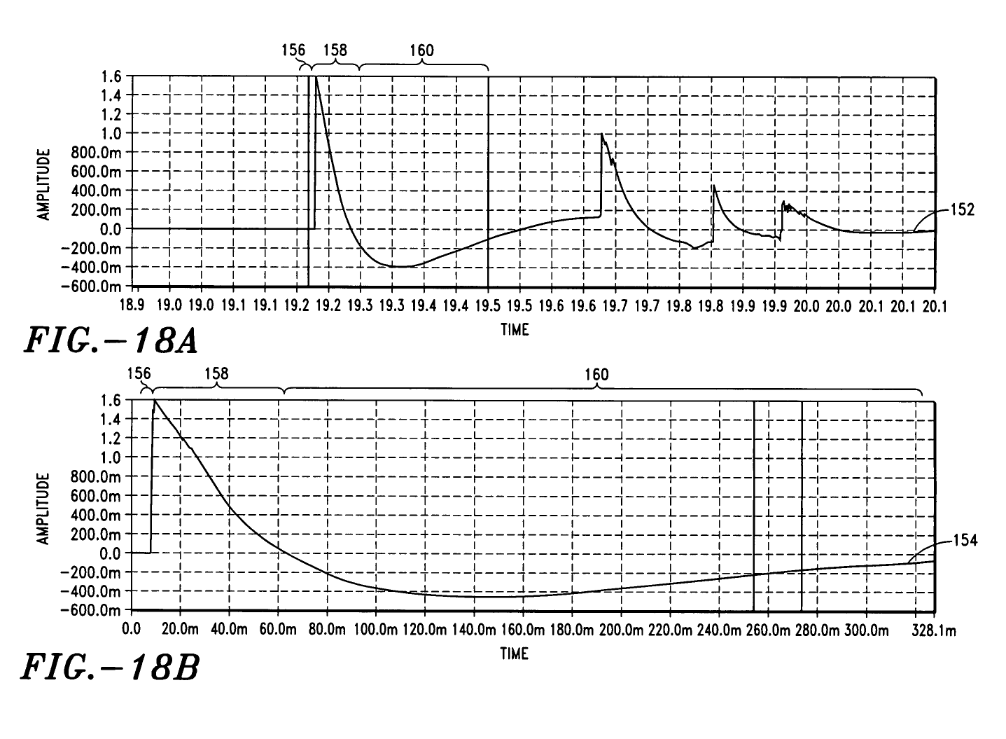

Referring now to FIGS. 17A-17B and 18A-18B, there are shown signals 148, 150, 152, 154 that were acquired from the radial nerve and are operative in the control of several muscles in the arm, wrist and fingers.

As illustrated in FIGS. 17A-17B and 18A-18B, each signal 148-154 includes a negative segment 156. It is believed that the negative segment 156 similarly reflects the nerve and/or muscle setting up for movement.

The spike or second segment 158 is the signal segment that moves the muscle. A longer spike segment 158 reflects a more pronounced muscle movement. The shorter segment 158 reflects quicker muscle movement. The higher the voltage during this segment 158, the stronger the muscle movement.

Following the spike or positive segment 158, is a negative segment 160. It is believed that this segment 160 reflects the rest and evaluation portion of the signal.

Referring now to FIGS. 18A-18B, there are shown signals 152, 154 that reflect the muscle responding to an environmental condition. More particularly, it is believed that the signals 152, 154 reflect the muscle moving in response to a sudden sharp pain. It can be seen that the second segment 158 is very strong. The third segment 160 is also more pronounced since the muscle had a greater movement and would require more rest.

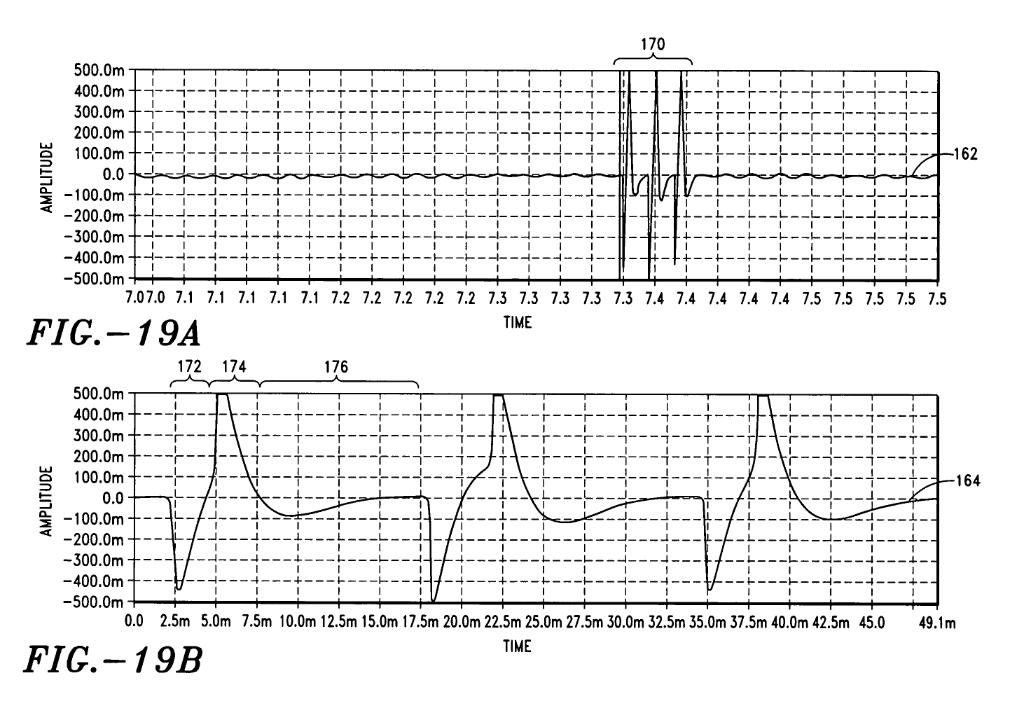

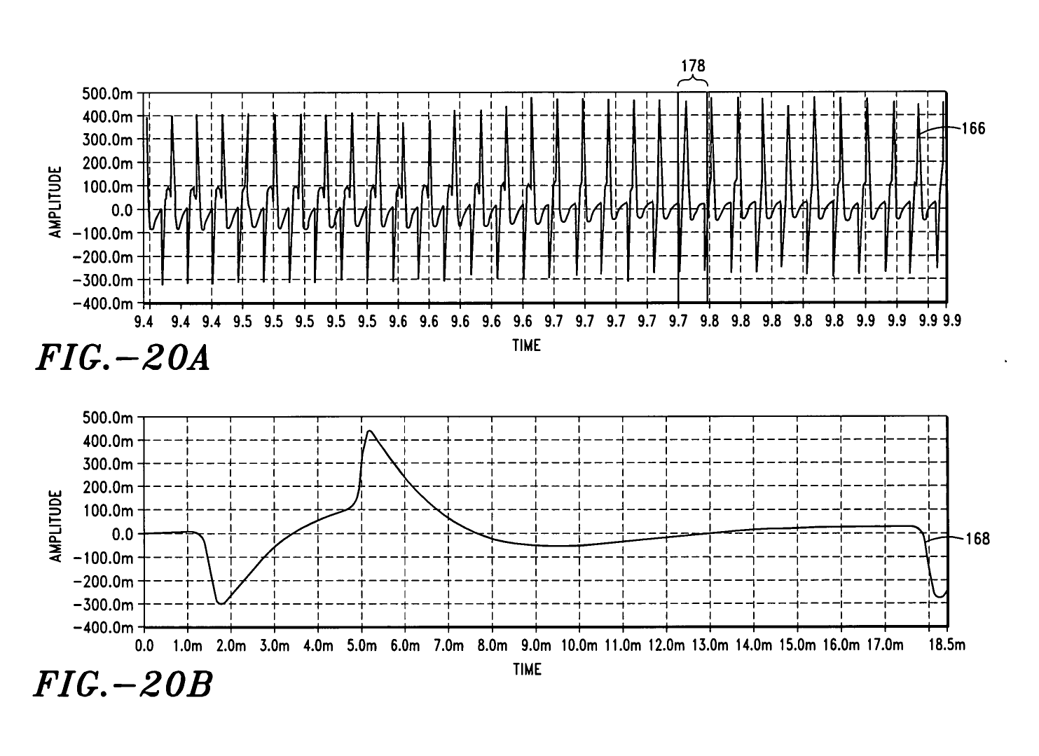

Referring now to FIGS. 19A-19B and 20A-20B, there are shown signals 162-168 acquired from the sciatic nerve that are operative in the control of several muscles in the leg, ankle and toes. Signal 164 reflects three movements. It can also be seen that the signals 162-168 similarly include a negative segment 172, and second positive segment 174, which produces movement in the muscle, and a third negative segment 176, which is believed to be the rest and evaluation portion of the signal.

Referring now to FIG. 20A, the signal 166 shows multiple leg movements. Segment 178 reflects a single leg movement, which is depicted in an expanded format in signal 168.

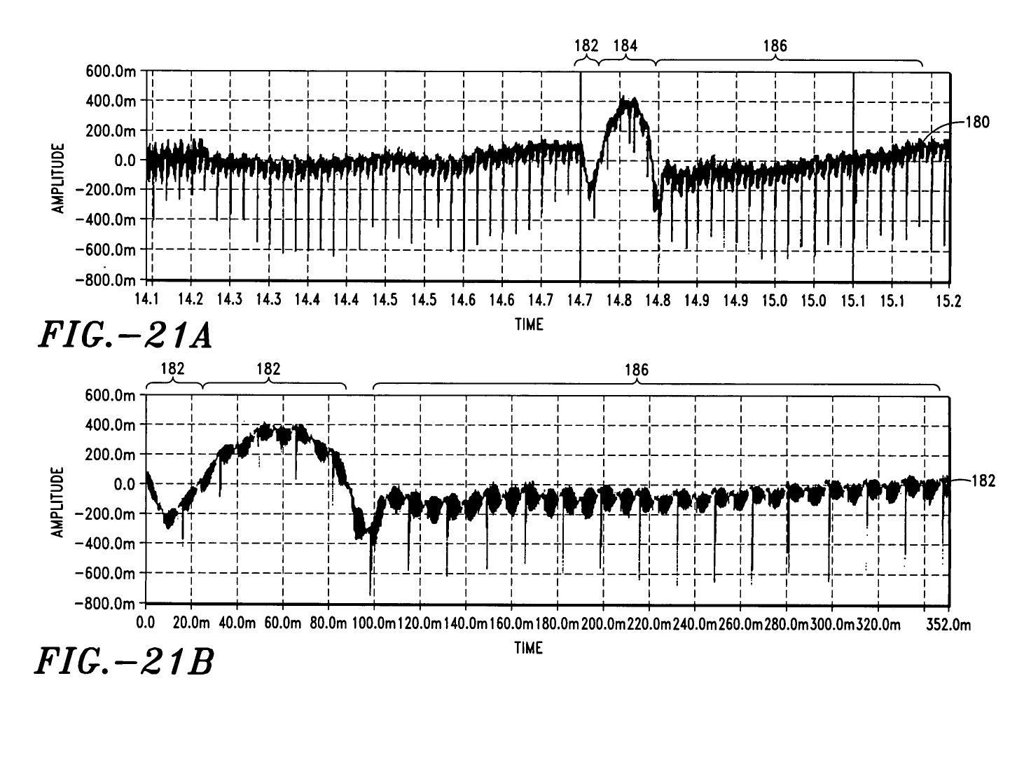

Referring now to FIGS. 21A and 21B there are shown signals 180, 182 acquired from the ulnar nerve that are operative in the control of several muscles in the arm, wrist and fingers. The noted signals 180, 182 similarly include a first negative segment 182, followed by a positive segment 184, which produces the required movement in the muscles and a third negative segment 186, reflecting the rest and evaluation portion of the signal.

Storage

Referring to FIG. 9, the converted signal is routed to the processing means of the invention. In a preferred embodiment of the invention, the processing means comprises a computer 240.

According to the invention, the computer 240 can include various operating systems. In a preferred embodiment, the computer includes a Windows® operating system.

Prior to capturing signal information, a unique directory is created on one of the computer disk drives to store the information to be captured. The directory name is then employed on the system configuration window in the directory field, which instructs the software where to store the captured data.

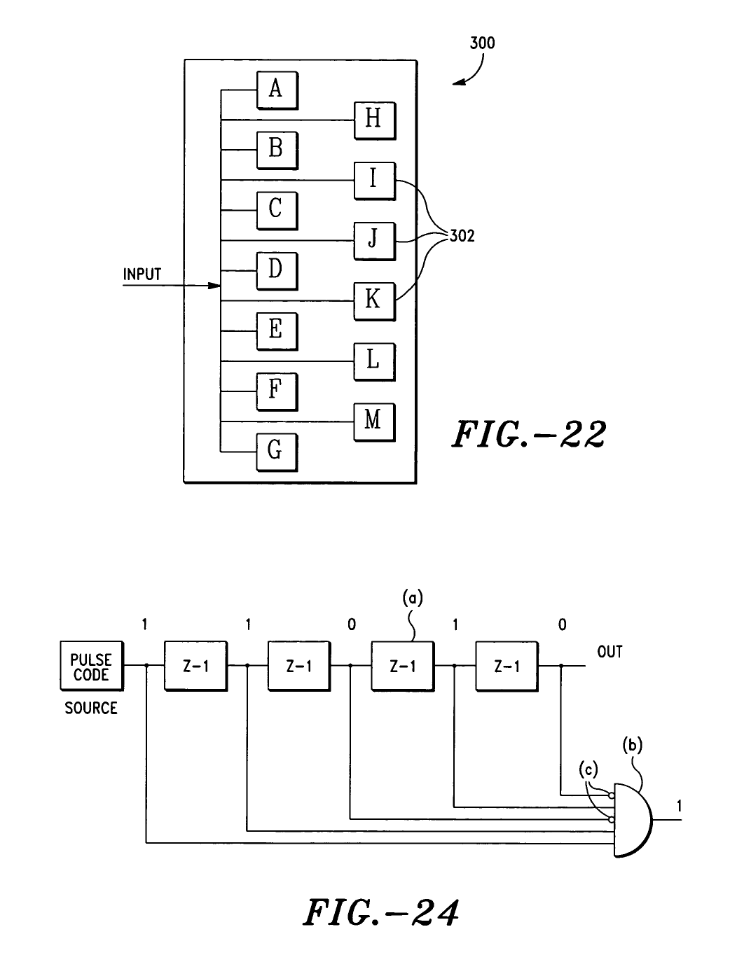

Referring now to FIG. 22, there is shown one embodiment of a storage module 300 of the programming means. As illustrated in FIG. 22, the storage module 300 includes a plurality of cells 302 (or files) that are adapted to receive at least one captured signal that is operative in the control of a target organ or muscle. By way of example, storage cell A can comprise captured signals operative in the control of the respiratory system; storage cell B can comprise captured signals operative in the control of the cardiovascular system, etc.

Preferably, the programming means of the invention is further adapted to store the captured signals according to the function performed by the signal. According to the invention, the noted signals can be stored separately within a designated storage cell 302 (e.g., storage cell A) or in a separate sub-cell.

According to the invention, the stored signals of each cell (e.g., A) and/or sub-cell can subsequently be employed to establish a base-line signal for each body function or organ. The computer can then be programmed to receive a plurality of signals from one or more probes, compare the signals to the golden signals to identify specific signals and store the identified signals in the appropriate cell 302.

In further envisioned embodiments of the invention, the computer is further programmed to compare “abnormal” signals captured from a subject and generate a modified base-line signal for transmission back to the subject. Such modification can include, for example, increasing the amplitude of a respiratory signal, increasing the rate of the signals, etc.

Transfer of Signals from Storage to Transmitting Means

Referring back to FIG. 9, to access a desired signal for transmission to a subject, one merely opens the file in the system. Once the desired signal is accessed, the user determines if frequency modulation (i.e., changes in amplitude/voltage) is necessary. If frequency modulation is desired or necessary, the user sets the modulation (e.g., 500 Hz) to provide the necessary signal modification.

In one embodiment of the invention, the modified (or unmodified) signal is then routed to a digital to analog converter 250 via lead 208 to convert the signals to an analog format. According to the invention, various digital to analog converters can be employed within the scope of the invention to provide the desired conversion. In a preferred embodiment, the converter 250 comprises a National Instruments DAQ Pad-6070E converter.

Transmission of Signals to the Subject

A key feature of the present invention is that the signals generated by the apparatus and methods described herein and transmitted to a subject are representative of the signals generated in the body. More particularly, the signal(s) transmitted to the subject substantially correspond to at least one waveform signal generated by the body and are operative in the control of at least one body organ (i.e., recognized by the brain or a selected organ as a modulation signal).

According to the invention, the signals generated by the processing means can be transmitted (or broadcast) to the subject by various conventional means (discussed in detail below). In a preferred embodiment, the signals are transmitted to the nervous system of the subject by direct conduction, i.e., direct engagement of a signal probe (or probes) to a target nerve. In alternative embodiments of the invention, the signals are transmitted externally via a signal probe (or probes) that is adapted to be in communication with the body (e.g., in contact with the body) and disposed proximate to a target nerve or selected organ.

Referring now to FIG. 9, in one embodiment of the invention, the converted waveform signal is routed from the digital to analog converter 250 to a biphasic stimulus isolator 260. The isolator unit 260 is adapted to isolate the signal sent to the body from the rest of the electronics.

The biphasic stimulus isolator 260 is preferably set to provide a constant current throughout the waveform signal. In a preferred embodiment, the varying voltages are preferably converted to percentages of + and −10 volts throughout the signal.

By way of example, if a specific point in the analog waveform signal equals 6 volts, then the percentage equals 60%. This percentage, i.e., 60%, is then used to calculate the current to be sent out. If the isolator 260 is set to an output range of 10 milliamps, then 60% results in 6 milliamps of output at that point in the analog waveform.

As the voltage of the analog waveform signal changes from zero to the maximum peak, the output from the isolator 260 will preferably have varying levels of current from zero to the corresponding percentage of the output range. The isolator 260 will thus ensure that the current being supplied is constant regardless of the changing resistance of the body.

In one embodiment of the invention, an oscilloscope is used to display the waveform signal transmitted from the isolator 260. The waveform signal shape should match what was displayed on the output window's graph. Indeed, the only possible change should be the amplitude or voltage of the waveform signals coming from the isolator 260.

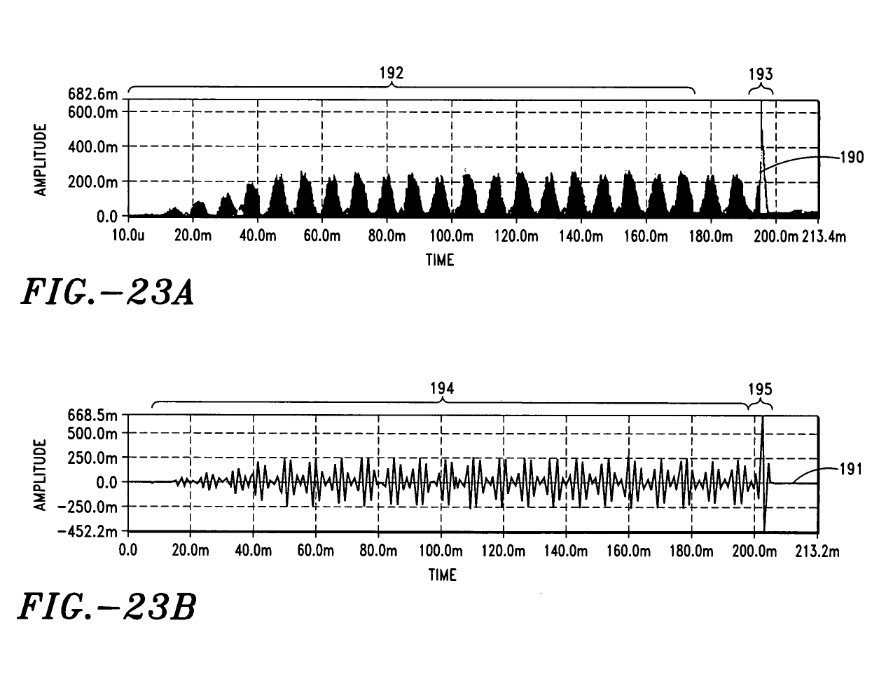

Referring now to FIGS. 23A and 23B, there are shown signals 190, 191 that were generated by the apparatus and methods of the invention. The noted signals are merely representative of the signals that can be generated by the apparatus and methods of the invention and should not be interpreted as limiting the scope of the invention in any way.

Referring first to FIG. 23A, there is shown the exemplar phrenic waveform signal 190 showing only the positive half of the transmitted signal. The signal 190 comprises only two segments, the initial segment 192 and the spike segment 193.

Referring now to FIG. 23B, there is shown the exemplar phrenic waveform signal 191 that has been fully modulated at 500 Hz. The signal 191 includes the same two segments, the initial segment 194 and the spike segment 195.

As is known in the art, the parameters for stimulating a nerve will change from nerve to nerve, organ to organ, and from human to human and animal to animal. Applicants have, however, found that a DC (direct current) voltage of over 2.5 volts can, and in many instances will, damage the phrenic nerve and an AC (alternating current) voltage of over 5 volts can, and in many instances will, contract the diaphragm muscle too much and cause pain and/or damage.

For proper stimulation of the target nerve of a human, in accordance with the invention, the amount of voltage of the waveform signal is thus preferably set to a low value. Preferably, the maximum transmitted voltage is in the range of 100 milli-volts-50 volts, more preferably, in the range of 100 milli-volts-5.0 volts, even more preferably, in the range of approximately 100-500 milli-volts (peak AC). In a preferred embodiment, the maximum transmitted voltage is less than 2 volts.

Preferably, the amperage is less than 2 amps, more preferably, in the range of 1 micro-amp-24 milli-amps, even more preferably, in the range of 1-1000 micro-amps. In a preferred embodiment, the amperage is in the range of 1-100 micro-amps.

As will be appreciated by one having ordinary skill in the art, it is also possible to develop a digital to analog conversion unit, which would provide enough electrical power to eliminate the need of the isolator 260. Care would however have to be exercised to ensure that this modified digital to analog conversion unit could also perform the function of isolating the body from the rest of the electronics.

In alternative embodiments of the invention, the analog to digital and digital to analog converters 220, 250 are eliminated. This is achieved by employing a pulse rate detector for input sampling and a pulse rate generator for output signal generation. The threshold for detection of pulses and the amplitude of generated pulses will be readily observed to be a direct function of the size of the nerve and the contact area of the electrodes employed.

In alternative embodiments, the functions described in the existing preferred embodiment of a laptop computer may be performed by utilizing discreet logic circuits, programmable logic arrays, microprocessors or microcontrollers, or Application Specific Integrated Circuits designed for the nerve detection and stimulus generation.

Referring back to FIG. 9, the waveform signal transmitted from the biphasic stimulus isolator 260 is routed to probes 270, 272. While probes 70, 72, which were employed to capture the signal, comprised simple hook probes, the probes for transmitting the signals of the invention can be varied depending on the size of the nerve.

For rat nerves, the hook probes are preferably still employed (with the signal probe cradling the target nerve and the ground probe attached to an interior muscle). The surgeon must however exercise extreme care when isolating the target nerve. The target nerve cannot be frayed, stretched too much, or twisted. Even slight damage will diminish the effect of the transmitted waveform signal.

For larger nerves (e.g., dog, pig, human), there are a variety of nerve probes that can be employed to transmit the signal(s) to the subject. By way of example, needle probes (e.g., World Precision Instruments PTM23B05) can be inserted into the target nerve. Nerve cuffs or spiral cuffs, which wrap around nerve forcing the electrodes to make contact with the target nerve, can also be employed.

Magnetic stimulation of nerves is also possible (e.g., Magstim Magstim 200). Transcutaneous electrical nerve stimulators (TENS) units, e.g., Bio Medical BioMed 2000, which magnetically stimulate the nerve through the skin, can also be employed. A laser can also be employed to stimulate the target nerve; or electromagnetic stimulation may be employed. Finally, ultrasonic and broadband transmission of the signals is also possible.

According to the invention, delivery of the waveform signal to the subject is not based upon a particular probe or probe design. Thus, a user can select a specific probe for a specific procedure.

Further, the transmitted signal can be transmitted to virtually any target nerve in the nervous system. Preferably, the signal is transmitted to a branch of the effector nerve proximal to divisional glanglia which branch to various portions of the target muscle or organ. In the case of the phrenic nerve, a preferred location is between the plexi in the neck and the diaphragm (shown generally as reference “79” in FIG. 8).

### EXAMPLES

The following examples are given to enable those skilled in the art to more clearly understand and practice the present invention. They should not be considered as limiting the scope of the invention, but merely as being illustrated as representative thereof.

### Example 1

A study was performed to locate the phrenic nerve in the neck and stimulate the diaphragm. A 0.58 kg rat was anesthetized; the neck, the back of the neck and chest were shaved. A tracheotomy was performed and the rat was intubated using a 14 g catheter. An incision was made at the back of the neck to locate the spine. A dremmel tool was used to perform a laminectomy and sever the spinal cord at C-2, C-3. Diaphragm and intercostal movement stopped.

The tracheotomy incision was extended to locate the right phrenic in the neck. The isoflurane was then reduced from 1 to 0.25% and the oxygen flow was then reduced to 0.3 L/min.

A hook probe was attached to the right phrenic nerve in the neck. The red (signal) lead was attached to the hook probe and the black (ground) lead was attached to an exposed muscle in the neck.

Stimulation began at 2:35 pm with strong diaphragm movement, and stopped at 9:35 pm. Throughout the seven hours, the rat was “breathing” using the input signal. As reflected in Table I, vital signs were within normal limits.

TABLE I

| | Heart Rate | | | | 02 Level | Degree of |
|---|---|---|---|---|---|---|
| Time | BPM | Blood Pressure | SPO2 | TEMP | L/min | Movement |
| 2:37 pm | 246 | | 98 | 96.4 | 0.3 | Strong |
| 2:45 pm | 246 | | 93 | 95.9 | 0.3 | Strong |
| 2:55 pm | 212 | 84/54 | 93 | 94.8 | 0.3 | Strong |
| 3:05 pm | 248 | 94/61 | 91 | 94.3 | 0.3 | Strong |
| 3:35 pm | 238 | 57/27 | 89 | 94.5 | 0.3 | Strong |
| 4:15 pm | 212 | 77/45 | 94 | 94.8 | 0.3 | Strong |
| 4:35 pm 2 hrs | 216 | 69/49 | 94 | 95 | 0.3 | Strong |
| 4:55 pm | 230 | 86/57 | 94 | 95.7 | 0.3 | Strong |
| 5:15 pm | 212 | 89/47 | 93 | 95.4 | 0.3 | Strong |
| 5:40 pm 3 hrs | 220 | 74/40 | 95 | 93.6 | 0.3 | Strong |
| 6:00 pm | 204 | 69/44 | 95 | 93.6 | 0.3 | Strong |
| 6:20 pm | 192 | 67/40 | 96 | 91 | 0.3 | Strong |
| 6:30 pm 4 hrs | 192 | 64/34 | 100 | 91.8 | 0.3 | Strong |
| 6:50 pm | 218 | 55/24 | 96 | 94.3 | 0.3 | Strong |
| 7:10 pm | 208 | 69/35 | 98 | 93.9 | 0.3 | Strong |
| | | SQ fluids to rat | | | | |
| 7:30 pm 5 hrs | 210 | 74/40 | 98 | 94.5 | 0.3 | Strong |
| 7:50 pm | 208 | 76/42 | 98 | 95.5 | 0.3 | Strong |
| 8:10 pm | 220 | 74/40 | 99 | 94.8 | 0.3 | Strong |
| 8:30 pm 6 hrs | 226 | 72/40 | 98 | 95 | 0.3 | Strong |
| 8:50 pm | 222 | 71/39 | 97 | 95.5 | 0.3 | Strong |
| 9:10 pm | 224 | 72/40 | 96 | 96.4 | 0.3 | Strong |
| 9:20 pm | 200 | 77/53 | 94 | 96.3 | 0.3 | Strong |
| 9:35 pm 7 hrs | 218 | Signal stopped | 87 | 96.3 | 0.3 | Strong |

### Example 2

A study was performed to locate the phrenic nerve in the neck and stimulate the diaphragm. A 0.74 kg rat was anesthetized; the neck, the back of the neck, and chest were shaved, a tracheotomy was performed. The rat was intubated using a 14 g catheter.

An incision was made at the back of the neck to locate the spine. A dremmel tool was used to perform a laminectomy and sever the spinal cord at C-2, C-3. Diaphragm and intercostals movement stopped.

The tracheotomy incision was extended to locate the right phrenic in the neck. The isoflurane was then reduced from 1 to 0.25% and the oxygen flow was reduced to 0.3 L/min.

A hook probe was attached to the right phrenic nerve in the neck. The red (signal) lead was attached to the hook probe and the black (ground) lead was attached to an exposed muscle in the neck. Stimulation began at 3:50 pm with strong diaphragm movement. At 4:05 pm the intercostals muscles began moving on their own again. Stimulation was stopped and another attempt was made to completely sever the spinal cord. Intercostal movement stopped. The probe was reattached to the right phrenic but no movement resulted when stimulated. The left phrenic was then located and the hook probe was attached.

Stimulation started at 4:30 pm with good strong diaphragm movement and continued until 7:30 pm when the study was ended. As reflected in Table II, vital signs were within the normal limits throughout the three hours that the rat was “breathing”.

TABLE II

| | Heart Rate | | | 02 Level | Degree of |
|---|---|---|---|---|---|
| Time | BPM | SPO2 | TEMP | L/min | Movement |
| 3:50 pm | 3.32 | 98 | 96.3 | 0.3 | Strong |
| 4:00 pm | 284 | 92 | 93.6 | 0.3 | Strong |
| 4:40 pm | 266 | 99 | 97 | 0.3 | Strong |
| 4:50 pm | 260 | 100 | 97.5 | 0.3 | Strong |
| 5:00 pm | 254 | 99 | 97.7 | 0.3 | Strong |
| 5:10 pm | 250 | 99 | 98.2 | 0.3 | Strong |
| 5:20 pm | 246 | 99 | 98.1 | 0.3 | Strong |
| 5:30 pm 1 hr | 250 | 99 | 98.2 | 0.3 | Strong |
| 5:40 pm | 236 | 98 | 98.1 | 0.3 | Strong |
| 5:50 pm | 242 | 100 | 97.7 | 0.25 | Strong |
| 6:00 pm | 242 | 98 | 97.5 | 0.25 | Strong |
| 6:20 pm | 234 | 97 | 97.9 | 0.25 | Strong |
| 6:30 pm 2 hrs | 238 | 98 | 97.5 | 0.25 | Strong |
| 6:40 pm | 242 | 98 | 97.5 | 0.25 | Strong |
| 6:50 pm | 240 | 98 | 97.5 | 0.25 | Strong |
| 7:00 pm | 232 | | 97.7 | 0.25 | Strong |
| 7:10 pm | 234 | 100 | 97.2 | 0.25 | Strong |
| 7:20 pm | 232 | 100 | 97 | 0.25 | Strong |
| 7:30 pm 3 hrs | 238 | 99 | 97 | 0.25 | Strong |

As will be appreciated by one having ordinary skill in the art, the method and system for recording, storing and transmitting waveform signals described above provides numerous advantages.

The method and systems of the invention can also be employed in numerous applications to control one or more body functions. Among the envisioned applications are the following:

a) Sleep Apnea

A patient is diagnosed with sleep apnea. A first sensor is employed to monitor diaphragm contractions, neck muscle tension, and/or airway pressure, and a second sensor is employed to capture signals from phrenic nerve or hypoglossal nerve. The signal(s) from the first sensor are routed to a processing unit (e.g., computer), where they are analyzed. If the signal indicates that a breath needs to be taken, a signal generated by the processing unit (as described herein) is transmitted to the subject to open the pharynx and/or contract the diaphragm.

b) Respiratory Distress

A patient is suffering from an inability to contract the diaphragm, e.g. from a high spinal cord injury. A first sensor is employed to monitor blood gas levels and a second sensor is employed to capture signals from the phrenic nerve. The signal(s) from the first sensor are routed to a processing unit (e.g., computer), where they are analyzed. If the signal indicates low blood oxygen levels, a signal generated by the processing unit (as described herein) is transmitted to the subject to contract the diaphragm.

c) Asthma

A patient is diagnosed with asthma. A first sensor is employed to monitor airway constriction and a second sensor is employed to capture signals from nerves innervating the bronchi and bronchioles. The signal(s) from the first sensor are routed to a processing unit (e.g., computer), where they are analyzed. If the signal indicates constricted airways, a signal generated by the processing unit (as described herein) is transmitted to the subject to open the constricted airways.

d) Low Blood Pressure

A patient is diagnosed with suffering from acute low blood pressure, e.g. as a result of traumatic blood loss or septic shock syndrome. A first sensor is employed to monitor blood pressure and a second sensor is employed to capture signals from the carotid sinus. The signal(s) from the first sensor are routed to a processing unit (e.g., computer), where they are analyzed. If the signal indicates low blood pressure, a signal generated by the processing unit (as described herein) is transmitted to the subject to increase blood pressure by constricting blood vessels.

e) Abnormal Heart Beat

A patient is diagnosed with an abnormal heart beat, e.g. atrial fibrillation, ventricular fibrillation, or tachycardia. A first sensor is employed to monitor the heart rate and a second sensor is employed to capture signals from nerves innervating the heart. The signal(s) from the first sensor are routed to a processing unit (e.g., computer), where they are analyzed. If the signal indicates an abnormal heart beat, a signal generated by the processing unit (as described herein) is transmitted to the subject to restore the heart to normal sinus rhythm.

f) Acid Reflux

A patient is diagnosed with acid reflux. A first sensor is employed to monitor acid levels in the lower esophagus and a second sensor is employed to capture muscle contraction signals from the lower esophageal sphincter. The signal(s) from the first sensor are routed to a processing unit (e.g., computer), where they are analyzed. If the signal indicates excess acid reflux, a signal generated by the processing unit (as described herein) is transmitted to the subject to tense the muscles of the lower esophageal sphincter.

g) Obesity

A patient is diagnosed with obesity. A first sensor is employed to monitor blood sugar levels and stomach contents and a second sensor is employed to capture signals from the vagus nerve. The signal(s) from the first sensor are routed to a processing unit (e.g., computer), where they are analyzed. If the signal indicates sufficient levels of blood sugar or that the stomach is sufficiently distended, a signal generated by the processing unit (as described herein) is transmitted to the subject to give a sensation of fullness and suppress the appetite.

h) Erectile Dysfunction

A patient is diagnosed with erectile dysfunction. A first sensor is employed to monitor penile tumescence and a second sensor is employed to capture signals from the dorsal penile nerve. The signal(s) from the first sensor are routed to a processing unit (e.g., computer), where they are analyzed. If the signal indicates erectile dysfunction, a signal generated by the processing unit (as described herein) is transmitted to the subject to achieve an erection.

Alternatively, a first sensor is employed to capture signals from the dorsal penile nerve. When an erection is desired but cannot be obtained naturally, a signal generated by the processing unit (as described herein) is transmitted to the subject to achieve an erection.

i) Stroke

A patient is diagnosed with a stroke that has affected motor control. A first sensor is employed to monitor muscle movement and a second sensor is employed to capture signals from the nerves innervating those muscles. The signal(s) from the first sensor are routed to a processing unit (e.g., computer), where they are analyzed. If the signal indicates inability to move, a signal generated by the processing unit (as described herein) is transmitted to the subject to move the muscles in order to maintain muscle tone.

Alternatively, a first sensor is employed to capture signals from the nerves innervating those muscles. If the patient is unable to move the desired muscles, a signal generated by the processing unit (as described herein) is transmitted to the subject to move the muscles in order to maintain muscle tone.

j) Tension Headaches

A patient is diagnosed with tension headaches. A first sensor is employed to monitor headache pain and a second sensor is employed to capture signals from the nerves innervating the muscles of the neck. The signal(s) from the first sensor are routed to a processing unit (e.g., computer), where they are analyzed. If the signal indicates a headache, a signal generated by the processing unit (as described herein) is transmitted to the subject to relax the muscles of the neck.

Alternatively, a first sensor is employed to capture signals from the nerves innervating the muscles of the neck. When the patient experiences a headache, a signal generated by the processing unit (as described herein) is transmitted to the subject to relax the muscles of the neck.

k) Weakened Immune System

A patient is immuno-compromised or is being immunized with a weak immunogen. A first sensor is employed to monitor immune function and a second sensor is employed to capture signals from thymus, lymph nodes, and/or spleen. The signal(s) from the first sensor are routed to a processing unit (e.g., computer), where they are analyzed. If the signal indicates a weakened immune system, a signal generated by the processing unit (as described herein) is transmitted to the subject to stimulate the immune response.

Alternatively, a first sensor is employed to capture signals from thymus, lymph nodes, and/or spleen. When the patient is being immunized with a weak immunogen or has a weakened immune system that needs to be bolstered, a signal generated by the processing unit (as described herein) is transmitted to the subject to stimulate the immune system.

l) Irritable Bowl Syndrome

A patient is diagnosed with irritable bowel syndrome. A first sensor is employed to monitor bowel contractions and a second sensor is employed to capture signals from the nerves innervating the bowel. The signal(s) from the first sensor are routed to a processing unit (e.g., computer), where they are analyzed. If the signal indicates abnormal bowel function, a signal generated by the processing unit (as described herein) is transmitted to the subject to restore normal bowel functions.

m) Low Sperm Count

A patient is diagnosed with low sperm count. A first sensor is employed to monitor sperm levels and a second sensor is employed to capture signals from nerves innervating the testes. The signal(s) from the first sensor are routed to a processing unit (e.g., computer), where they are analyzed. If the signal indicates low sperm count, a signal generated by the processing unit (as described herein) is transmitted to the subject to increase production of sperm.

Alternatively, a first sensor is employed to capture signals from nerves innervating the testes. In order to increase the sperm count, a signal generated by the processing unit (as described herein) is transmitted to the subject to increase production of sperm.

n) Muscle Cramps

A patient is diagnosed with muscle cramps. A first sensor is employed to monitor muscle conditions and a second sensor is employed to capture signals from the nerves innervating those muscles. The signal(s) from the first sensor are routed to a processing unit (e.g., computer), where they are analyzed. If the signal indicates a muscle cramp, a signal generated by the processing unit (as described herein) is transmitted to the subject to relax the cramped muscle.

Alternatively, a first sensor is employed to capture signals from the nerves innervating those muscles. When the patient experiences a muscle cramp, a signal generated by the processing unit (as described herein) is transmitted to the subject to relax the cramped muscle.

o) Sexual Unresponsiveness

A patient is suffering from an inability to achieve orgasm. A first sensor is employed to monitor whether an orgasm has been achieved and a second sensor is employed to capture signals from the external genitalia responsible for orgasm. The signal(s) from the first sensor are routed to a processing unit (e.g., computer), where they are analyzed. If the signal indicates a lack of orgasm after an appropriate length of time, a signal generated by the processing unit (as described herein) is transmitted to the subject to achieve orgasm.

p) Insomnia

A patient is diagnosed with insomnia. A first sensor is employed to monitor fatigue and a second sensor is employed to capture signals from large muscle groups. The signal(s) from the first sensor are routed to a processing unit (e.g., computer), where they are analyzed. If the signal indicates a lack of sleep, a signal generated by the processing unit (as described herein) is transmitted to the subject to relax their large muscle groups and aid in falling asleep.

Alternatively, a first sensor is employed to capture signals from large muscle groups. If the patient is unable to fall asleep, a signal generated by the processing unit (as described herein) is transmitted to the subject to relax his large muscle groups and aid in falling asleep.

q) Restless Legs Syndrome

A patient is diagnosed with restless legs syndrome. A first sensor is employed to monitor leg movement and a second sensor is employed to capture signals from the nerves innervating the muscles of the legs. The signal(s) from the first sensor are routed to a processing unit (e.g., computer), where they are analyzed. If the signal indicates restless legs, a signal generated by the processing unit (as described herein) is transmitted to the subject to relax the muscles of the legs.

r) Incontinence

A patient is diagnosed with urinary incontinence. A first sensor is employed to monitor fullness of the bladder and a second sensor is employed to capture signals from the urethral sphincter. The signal(s) from the first sensor are routed to a processing unit (e.g., computer), where they are analyzed. If the signal indicates a less than full bladder, a signal generated by the processing unit (as described herein) is transmitted to the subject to keep the sphincter closed. When the bladder needs to be emptied, at the appropriate moment, a signal is transmitted to open the sphincter.

s) Constipation

A patient is suffering from constipation. A first sensor is employed to monitor bowel movement and a second sensor is employed to capture signals from nerves innervating the bowel. The signal(s) from the first sensor are routed to a processing unit (e.g., computer), where they are analyzed. If the signal indicates constipation, a signal generated by the processing unit (as described herein) is transmitted to the subject to increase peristaltic motion.

t) Nausea

A patient is suffering from frequent nausea. A first sensor is employed to monitor levels of nausea and a second sensor is employed to capture signals from the vagus nerve. The signal(s) from the first sensor are routed to a processing unit (e.g., computer), where they are analyzed. If the signal indicates nausea, a signal generated by the processing unit (as described herein) is transmitted to the subject to counteract the signals of nausea.

u) Spasticity

A patient is diagnosed with spasticity. A first sensor is employed to monitor muscle tension and a second sensor is employed to capture signals from the nerves that innervate the muscles. The signal(s) from the first sensor are routed to a processing unit (e.g., computer), where they are analyzed. If the signal indicates continuously contracted muscles, a signal generated by the processing unit (as described herein) is transmitted to the subject to relax the muscles.

v) Dry Eyes Syndrome

A patient is diagnosed with dry eyes syndrome. A first sensor is employed to monitor levels of tears and a second sensor is employed to capture signals from the nerves innervating the lacrimal glands. The signal(s) from the first sensor are routed to a processing unit (e.g., computer), where they are analyzed. If the signal indicates dry eyes, a signal generated by the processing unit (as described herein) is transmitted to the subject to increase tear production.

w) Dry Mouth Syndrome

A patient is diagnosed with dry mouth syndrome. A first sensor is employed to monitor levels of saliva and a second sensor is employed to capture signals from the nerves innervating the salivary glands. The signal(s) from the first sensor are routed to a processing unit (e.g., computer), where they are analyzed. If the signal indicates dry mouth, a signal generated by the processing unit (as described herein) is transmitted to the subject to increase saliva production and secretion.

x) Depression

A patient is diagnosed with depression. A first sensor is employed to monitor signals from the limbic system and a second sensor is employed to capture signals from the vagus nerve. The signal(s) from the first sensor are routed to a processing unit (e.g., computer), where they are analyzed. If the signal indicates a depressed mood, a signal generated by the processing unit (as described herein) is transmitted to the subject to produce a state of euphoria.

y) Epilepsy

A patient is diagnosed with epilepsy. A first sensor is employed to monitor brain wave activity and a second sensor is employed to capture signals from the vagus nerve. The signal(s) from the first sensor are routed to a processing unit (e.g., computer), where they are analyzed. If the signal indicates an epileptic attack is imminent, a signal generated by the processing unit (as described herein) is transmitted to the subject to counteract the epileptic attack.

z) Overactive Bladder

A patient is diagnosed with an overactive bladder. A first sensor is employed to monitor the status of the bladder and a second sensor is employed to capture signals from nerves that innervate the bladder. The signal(s) from the first sensor are routed to a processing unit (e.g., computer), where they are analyzed. If the signal indicates an overactive bladder, a signal generated by the processing unit (as described herein) is transmitted to the subject to relax the muscles of the bladder.

aa) Low Levels of Growth Hormone

A patient is diagnosed with low levels of growth hormone. A first sensor is employed to monitor growth hormone levels and a second sensor is employed to capture signals from the pituitary gland. The signal(s) from the first sensor are routed to a processing unit (e.g., computer), where they are analyzed. If the signal indicates low levels of growth hormone, a signal generated by the processing unit (as described herein) is transmitted to the subject to increase growth hormone levels.

bb) Low Levels of Insulin

A patient is diagnosed with low levels of insulin. A first sensor is employed to monitor insulin and blood sugar levels and a second sensor is employed to capture signals from the pancreas. The signal(s) from the first sensor are routed to a processing unit (e.g., computer), where they are analyzed. If the signal indicates low levels of insulin and high levels of blood sugar, a signal generated by the processing unit (as described herein) is transmitted to the subject to increase insulin secretion.

cc) Abnormal Levels of Thyroid Hormone

A patient is diagnosed with abnormal levels of thyroid hormone. A first sensor is employed to monitor thyroid hormone levels and a second sensor is employed to capture signals from the thyroid and/or the pituitary gland. The signal(s) from the first sensor are routed to a processing unit (e.g., computer), where they are analyzed. If the signal indicates abnormal levels of thyroid hormone, a signal generated by the processing unit (as described herein) is transmitted to the subject to restore the thyroid hormone levels to normal, either by increasing or decreasing secretion of thyroid hormone or thyroid stimulating hormone, as appropriate.

dd) Abnormal Levels of Melatonin

A patient is diagnosed with abnormal levels of melatonin. A first sensor is employed to monitor melatonin levels and a second sensor is employed to capture signals from the pineal gland. The signal(s) from the first sensor are routed to a processing unit (e.g., computer), where they are analyzed. If the signal indicates abnormal levels of melatonin, a signal generated by the processing unit (as described herein) is transmitted to the subject to restore the melatonin levels to normal, either by increasing or decreasing secretion of the hormone, as appropriate.

ee) Abnormal Levels of Adrenocorticotrophic Hormone

A patient is diagnosed with abnormal levels of adrenocorticotrophic hormone (ACTH). A first sensor is employed to monitor levels of ACTH and a second sensor is employed to capture signals from the pituitary gland. The signal(s) from the first sensor are routed to a processing unit (e.g., computer), where they are analyzed. If the signal indicates an abnormal level of ACTH, a signal generated by the processing unit (as described herein) is transmitted to the subject to restore the ACTH levels to normal, either by increasing or decreasing secretion of the hormone, as appropriate.

ff) Abnormal Levels of Antidiuretic Hormone (ADH)

A patient is diagnosed with abnormal levels of antidiuretic hormone (ADH). A first sensor is employed to monitor levels of ADH and a second sensor is employed to capture signals from the pituitary gland. The signal(s) from the first sensor are routed to a processing unit (e.g., computer), where they are analyzed. If the signal indicates abnormal levels of ADH, a signal generated by the processing unit (as described herein) is transmitted to the subject to restore the ADH levels to normal, either by increasing or decreasing secretion of the hormone, as appropriate.

gg) Abnormal Levels of Parathyroid Hormone

A patient is diagnosed with abnormal levels of parathyroid hormone. A first sensor is employed to monitor levels of parathyroid hormone and a second sensor is employed to capture signals from the parathyroid glands. The signal(s) from the first sensor are routed to a processing unit (e.g., computer), where they are analyzed. If the signal indicates abnormal levels of parathyroid hormone, a signal generated by the processing unit (as described herein) is transmitted to the subject to restore the parathyroid hormone levels to normal, either by increasing or decreasing secretion of the hormone, as appropriate.

hh) Abnormal Levels of Epinephrine or Norepinephrine

A patient is diagnosed with abnormal levels of epinephrine or norepinephrine. A first sensor is employed to monitor levels of epinephrine or norepinephrine and a second sensor is employed to capture signals from the adrenal glands. The signal(s) from the first sensor are routed to a processing unit (e.g., computer), where they are analyzed. If the signal indicates abnormal levels of epinephrine or norepinephrine, a signal generated by the processing unit (as described herein) is transmitted to the subject to restore the epinephrine or norepinephrine levels to normal, either by increasing or decreasing secretion of the hormone, as appropriate.

ii) Abnormal Levels of Glucagon

A patient is diagnosed with abnormal levels of glucagon. A first sensor is employed to monitor levels of glucagon and a second sensor is employed to capture signals from the pancreas. The signal(s) from the first sensor are routed to a processing unit (e.g., computer), where they are analyzed. If the signal indicates abnormal levels of glucagon, a signal generated by the processing unit (as described herein) is transmitted to the subject to restore glucagon levels to normal, either by increasing or decreasing secretion of the hormone, as appropriate.

jj) Abnormal Levels of Sex Hormones

A patient is diagnosed with abnormal levels of sex hormones, e.g., testosterone or estrogen. A first sensor is employed to monitor levels of sex hormones and a second sensor is employed to capture signals from the gonads. The signal(s) from the first sensor are routed to a processing unit (e.g., computer), where they are analyzed. If the signal indicates abnormal levels of sex hormones, a signal generated by the processing unit (as described herein) is transmitted to the subject to restore sex hormone levels to normal, either by increasing or decreasing secretion of the hormone, as appropriate.

kk) Pain Abatement

A patient is suffering from chronic pain. A first sensor is employed to monitor pain signals and a second sensor is employed to capture signals from the relevant nerve. The signal(s) from the first sensor are routed to a processing unit (e.g., computer), where they are analyzed. If the signal indicates pain, a signal generated by the processing unit (as described herein) is transmitted to the subject to counteract the pain signal.

A patient is preparing to undergo a procedure that will produce pain, e.g., surgery, a dental extraction or childbirth. A first sensor is employed to capture signals from the relevant nerve, e.g. the trigeminal nerve. A signal generated by the processing unit (as described herein) is transmitted to the subject to block the sensation of pain from the procedure.

ll) Organ Transplant

A patient is undergoing heart or liver transplant, and ice is placed inside the body during the procedure. Following the procedure, a first sensor is employed to capture signals from the phrenic nerve. The signals are compared to those of normal phrenic nerves to diagnose any damage done to the nerve during the procedure.

mm) Paralysis

A patient has suffered a stroke and some muscles are not moveable. A first sensor is employed to capture signals from the nerves that innervate the paralyzed muscle. The signals are compared to those from normal nerves to diagnose any damage done to the nerve from the stroke.

nn) Cardiac Irregularity

A patient suffers a cardiac irregularity. A first sensor is employed to capture signals from the heart. The signals are compared to those from normal heart to diagnose any damage done to the heart and evaluate its condition.

oo) Spinal Chord Injury

A patient suffers a spinal cord injury. A first sensor is employed to capture signals from various nerves that emerge from the spinal cord. The signals are compared to those from normal nerves to diagnose any damage done to the nerves.

pp) Physical Therapy

A patient has undergone surgery, e.g., hip replacement, knee surgery, etc. A first sensor is employed to capture signals to the affected muscles. A signal generated by the processing unit (as described herein) is transmitted to the subject to move the affected muscles and provide physical therapy.

qq) Deep Tissue Injury

A patient has suffered a deep tissue injury. A first sensor is employed to capture signals to the affected deep tissue. A signal generated by the processing unit (as described herein) is transmitted to the subject to provide increased blood flow to the affected deep tissue.

rr) Military Interrogation

A military, government, or law enforcement agency desires a non-lethal weapon to subdue or interrogate an opponent. A signal generated by the processing unit (as described herein) is transmitted to the subject to subdue said subject. This signal may include, but is not limited to, causing the bladder or bowel to evacuate, causing temporary blindness, causing a temporary ringing in the ears, causing hyperventilation to the point of losing consciousness, or causing temporary severe pain.

ss) Traumatic Injury

A patient has suffered a traumatic injury, and trained emergency medical personnel need to provide immediate treatment to manage the patient. A signal generated by the processing unit (as herein described) is transmitted to the subject to stabilize vital signs or provide support. Examples of transmitted signals include a signal to control breathing frequency and volume, a signal to control heart rate, a signal to regulate blood pressure, a signal to reduce pain, or a signal to induce unconsciousness.

tt) Alternative to “Chemical Castration”

A patient requiring suppression of sexual desire to affect re-entry to society following sexual abuse treatment may have the regulation of sexual hormones controlled by signals applied to nerve connections to the hormone secretion glands to reduce testosterone levels without excessive generation of estrogen.

uu) Muscle Atrophy

A patient is in a coma. A first sensor is employed to capture signals from relevant muscle groups. If it is desired that the patient receive muscle stimulation, a signal generated by the processing unit (as described herein) is transmitted to the subject to contract his muscles regularly to aid in maintaining muscle tone.

vv) Acupuncture

A patient is undergoing acupuncture treatment. A first sensor is employed to capture signals from the relevant body organ being treated. If it is desired that the patient receive electrical stimulation through the acupuncture needles, a signal generated by the processing unit (as described herein) is transmitted to the subject to achieve the desired acupuncture treatment.

ww) Chiropractic

A patient is undergoing chiropractic treatments. A first sensor is employed to capture signals from the relevant body organ being treated. If it is desired that the patient receive electrical stimulation in conjunction with chiropractic treatment, a signal generated by the processing unit (as described herein) is transmitted to the subject to achieve the desired chiropractic treatment.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

1. A method for regulating body organ function, comprising the steps of:
capturing a plurality of waveform signals generated in a subject's body, said waveform signals being operative in the regulation of body organ function; and
transmitting at least a first waveform signal to said body, said first waveform signal being recognizable by at least one body organ as a modulation signal.

2. The method of claim 1, wherein said first waveform signal is transmitted to said subject's nervous system.

3. The method of claim 1, wherein said subject comprises a human.

4. The method of claim 1, wherein said subject comprises an animal.

5. A method for regulating body organ function, comprising the steps of:
capturing a plurality of waveform signals generated in a subject's body, said waveform signals being operative in the regulation of body organ function; and
transmitting at least a first waveform signal proximate a first body organ to regulate organ function, said first waveform signal including at least a second waveform signal that substantially corresponds to at least one of said captured waveform signals and is operative in the regulation of said first body organ.

6. The method of claim 5, wherein said first waveform signal is transmitted to said subject's nervous system.

7. The method of claim 5, wherein said subject comprises a human.

8. The method of claim 5, wherein said subject comprises an animal.

9. A method for regulating body organ function, comprising the steps of:
capturing a plurality of waveform signals generated in a subject's body, said waveform signals being operative in the regulation of body organ function;
storing said captured waveform signals in a storage medium, said storage medium being adapted to store said captured waveform signals according to the organ regulated by said captured waveform signals; and
transmitting at least a first waveform signal proximate a first body organ to regulate organ function, said first waveform signal including at least a second waveform signal that substantially corresponds to at least one of said captured waveform signals and is operative in the regulation of said first body organ.

10. The method of claim 9, wherein said first waveform signal is transmitted to said subject's nervous system.

11. The method of claim 9, wherein said storage medium is further adapted to store said captured waveform signals according to the function performed by said captured waveform signals.

12. A method for regulating body organ function, comprising the steps of:
capturing a plurality of waveform signals generated in a subject's body, said waveform signals including at least a first waveform signal that is operative in the control of at least one body organ;
storing said captured waveform signals in a storage medium, said storage medium being adapted to store said captured waveform signals according to the organ regulated by said captured waveform signals;
generating at least a second waveform signal, said second waveform signal substantially corresponding to at least said first waveform signal and is operative in the regulation of said body organ;
transmitting said second waveform signal proximate said body organ to regulate organ function.

13. The method of claim 12, wherein said second waveform signal is transmitted to said subject's nervous system.

14. The method of claim 12, wherein said storage medium is further adapted to store said captured waveform signals according to the function performed by said waveform signals.

15. A method for regulating body organ function, comprising the steps of:
capturing a first plurality of waveform signals generated in a first subject's body, said first plurality of waveform signals including first waveform signals that are operative in the control of a first body organ;
generating a base-line waveform signal from said first waveform signals;
capturing a second plurality of waveform signals generated in said first subject's body, said second plurality of waveform signals including at least a second waveform signal that is operative in the control of said first body organ;
comparing said base-line waveform signal to said second waveform signal;
generating a third waveform signal based on said comparison of said base-line and second waveform signals;
transmitting said third waveform signal proximate said first body organ, said third waveform signal being operative in the regulation of said first organ function.

16. The method of claim 15, wherein said step of capturing said waveform signals comprises capturing said first plurality of waveform signals from a plurality of subjects.

17. The method of claim 15, wherein said third waveform substantially corresponds to said second waveform signal.

18. The method of claim 15, wherein said third waveform substantially corresponds to said base-line waveform signal.

19. The method of claim 15, wherein said third waveform signal is transmitted to said subject's nervous system.

20. The method of claim 15, wherein said subject comprises a human.

21. The method of claim 15, wherein said subject comprises an animal.

22. A method for regulating body organ function, comprising the steps of:
capturing a first plurality of waveform signals generated in a first subject's body, said first plurality of waveform signals including first waveform signals that are operative in the control of a first body organ;
storing said first waveform signals in a first location in a storage medium;
generating a base-line waveform signal from said first waveform signals;
capturing a second plurality of waveform signals generated in said first subject's body, said second plurality of waveform signals including at least a second waveform signal that is operative in the control of said first body organ;
storing said second waveform signal in a second location in said storage medium;
comparing said base-line waveform signal to said second waveform signal;
generating a third waveform signal based on said comparison of said base-line and second waveform signals;
transmitting said third waveform signal proximate said first body organ, said third waveform signal being operative in the regulation of said first organ function.

23. The method of claim 22, wherein said step of capturing said waveform signals comprises capturing said first plurality of waveform signals from a plurality of subjects.

24. The method of claim 22, wherein said third waveform signal is transmitted to said subject's nervous system.

25. The method of claim 22, wherein said subject comprises a human.

26. The method of claim 22, wherein said subject comprises an animal.

27. A system for regulating body organ function, comprising:
at least a first signal probe adapted to capture waveform signals from a subject's body, said waveform signals being representative of waveform signals naturally generated in said body and indicative of body organ function;
a processor in communication with said signal probe and adapted to receive said waveform signals, said processor being further adapted to generate at least a first waveform signal based on said captured waveform signals, said first waveform signal being recognizable by at least one body organ as a modulation signal; and
at least a second signal probe adapted to be in communication with said subject's body for transmitting said first waveform signal proximate to said body organ to regulate organ function.

28. The system of claim 27, wherein said processor includes a pulse rate detector for sampling said captured waveform signals.

29. The system of claim 28, wherein said processor includes a pulse rate generator for generating said first waveform signal.

30 The system of claim 27, wherein said processor includes a storage medium adapted to store said captured waveform signals.

31. The system of claim 30, wherein said storage medium is adapted to store said captured waveform signals according to the organ regulated by said captured waveform signals.

32. The system of claim 31, wherein said storage medium is further adapted to store said captured waveform signals according to the function performed by said captured waveform signals.

33. The system of claim 27, wherein said second signal probe is adapted to transmit said first waveform signal directly to said subject by direct conduction to the subject's nervous system.
